(19) United States
(12) Patent Application Publication
(10) Pub. No.: US 2024/0360067 A1
(43) Pub. Date: Oct. 31, 2024

(54) DERIVATIVES OF ITACONIC ACID AND THEIR USE AS ANTI-INFLAMMATORY AGENTS

(71) Applicant: Sitryx Therapeutics Limited, Oxford (GB)

(72) Inventors: Michael Liam COOKE, Nottingham, Nottinghamshire (GB); Matthew Colin Thor FYFE, Oxford (GB); Alessandro MAZZACANI, Oxford (GB)

(73) Assignee: Sitryx Therapeutics Limited, Oxford (GB)

(21) Appl. No.: 18/682,780

(22) PCT Filed: Aug. 11, 2022
(86) PCT No.: PCT/GB2022/052090

§ 371 Date: Feb. 9, 2024

(30) Foreign Application Priority Data
Aug. 11, 2021 (EP) 21190883.5

(51) Int. Cl. C07C 69/618; A61K 31/225; A61K 31/337; A61K 31/351; A61K 31/38; A61K 31/381; A61K 31/4164; A61K 31/4418; A61K 31/445; A61K 31/5375; C07C 235/76; C07C 237/16; C07D 211/58; C07D 213/55; C07D 233/64; C07D 295/15; C07D 305/08; C07D 309/14; C07D 331/04; C07D 333/24

(52) U.S. Cl. CPC C07C 69/618; A61K 31/225; A61K 31/337; A61K 31/351; A61K 31/38; A61K 31/381; A61K 31/4164; A61K 31/4418; A61K 31/445; A61K 31/5375; C07C 235/76; C07C 237/16; C07D 211/58; C07D 213/55; C07D 233/64; C07D 295/15; C07D 305/08; C07D 309/14; C07D 331/04; C07D 333/24; C07C 2601/04; C07C 2601/16

(57) ABSTRACT

The invention relates to compounds of formula (I) and to their use in treating or preventing an inflammatory disease or a disease associated with an undesirable immune response:

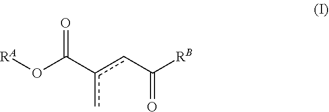

wherein R^{A} and R^{B} are as defined herein.

### FIELD OF THE INVENTION

The present invention relates to compounds and their use in treating or preventing inflammatory diseases or diseases associated with an undesirable immune response, and to related compositions, methods and intermediate compounds.

### BACKGROUND OF THE INVENTION

Chronic inflammatory diseases such as rheumatoid arthritis, systemic lupus erythematosus (SLE), multiple sclerosis, psoriasis, Crohn's disease, ulcerative colitis, uveitis and chronic obstructive pulmonary disease (COPD) represent a significant burden to society because of life-long debilitating illness, increased mortality and high costs for therapy and care (Straub R. H. and Schradin C., 2016). Non-steroidal anti-inflammatory drugs (NSAIDs) are the most widespread medicines employed for treating inflammatory disorders, but these agents do not prevent the progression of the inflammation and only treat the accompanying symptoms. Glucocorticoids are powerful anti-inflammatory agents, making them emergency treatments for acute inflammatory flares, but given longer term these medicines give rise to a plethora of unwanted side-effects and may also be subject to resistance (Straub R. H. and Cutolo M., 2016). Thus, considerable unmet medical need still exists for the treatment of inflammatory disorders and extensive efforts to discover new medicines to alleviate the burden of these diseases is ongoing (Hanke T. et al., 2016).

Dimethyl fumarate (DMF), a diester of the citric acid cycle (CAC) intermediate fumaric acid, is utilised as an oral therapy for treating psoriasis (Bruck J. et al., 2018) and multiple sclerosis (Mills E. A. et al., 2018). Importantly, following oral administration, none of this agent is detected in plasma (Dibbert S. et al., 2013), the only drug-related compounds observed being the hydrolysis product monomethyl fumarate (MMF) and glutathione (GSH) conjugates of both the parent (DMF) and metabolite (MMF). DMF's mechanism of action is complex and controversial. This compound's efficacy has been attributed to a multiplicity of different phenomena involving covalent modification of proteins and the conversion of “prodrug” DMF to MMF. In particular, the following pathways have been highlighted as being of relevance to DMF's anti-inflammatory effects: 1) activation of the anti-oxidant, anti-inflammatory, nuclear factor (erythroid-derived 2)-like 2 (NRF2) pathway as a consequence of reaction of the electrophilic α,β-unsaturated ester moiety with nucleophilic cysteine residues on kelch-like ECH-associated protein 1 (KEAP1) (Brennan M. S. et al., 2015); 2) induction of activating transcription factor 3 (ATF3), leading to suppression of pro-inflammatory cytokines interleukin (IL)-6 and IL-8 (Müller S. et al., 2017); 3) inactivation of the glycolytic enzyme glyceraldehyde 3-phosphate dehydrogenase (GAPDH) through succination of its catalytic cysteine residue with a Michael accepting unsaturated ester (Kornberg M. D. et al., 2018; Angiari S. and O'Neill L. A., 2018); 4) inhibition of nuclear factor-kappaB (NF-κB)-driven cytokine production (Gillard G. O. et al., 2015); 5) preventing the association of PKCθ with the costimulatory receptor CD28 to reduce the production of IL-2 and block T-cell activation (Blewett M. M. et al., 2016); 6) reaction of the electrophilic α,β-unsaturated ester with the nucleophilic thiol group of anti-oxidant GSH, impacting cellular responses to oxidative stress (Lehmann J. C. U. et al., 2007); 7) agonism of the hydroxycarboxylic acid receptor 2 (HCA2) by the MMF generated in vivo through DMF hydrolysis (von Glehn F. et al., 2018); 8) allosteric covalent inhibition of the p90 ribosomal S6 kinases (Andersen J. L. et al., 2018); 9) inhibition of the expression and function of hypoxia-inducible factor-1a (HIF-1a) and its target genes, such as IL-8 (Zhao G. et al., 2014); and 10) inhibition of Toll-like receptor (TLR)-induced M1 and K63 ubiquitin chain formation (McGuire V. A. et al., 2016). In general, with the exception of HCA2 agonism (Tang H. et al., 2008), membrane permeable diester DMF tends to exhibit much more profound biological effects in cells compared to its monoester counterpart MMF. However, the lack of systemic exposure of DMF in vivo has led some researchers to assert that MMF is, in fact, the principal active component following oral DMF administration (Mrowietz U. et al., 2018). As such, it is evident that some of the profound biology exerted by DMF in cells is lost because of hydrolysis in vivo to MMF.

Recently, it has been discovered that, during inflammatory macrophage activation, the CAC becomes anaplerotic and is diverted such that the unsaturated diacid itaconic acid, “itaconate”, is generated (Murphy M. P. and O'Neill L. A. J., 2018; O'Neill L. A. J. and Artyomov M. N., 2019; Yu X.-H. et al., 2019). Instead of being hydrated to isocitrate by aconitate hydratase, the CAC intermediate aconitate is decarboxylated by the protein product of immune-responsive gene 1 (IRG1), one of the most highly upregulated genes in macrophages under proinflammatory conditions, subsequently named aconitate decarboxylase 1, to produce itaconic acid (Michelucci A. et al., 2013). This unsaturated diacid is an inhibitor of the bacterial enzyme isocitrate lyase and, as such, it exerts anti-bacterial activity. In addition, itaconic acid has been shown to inhibit the CAC enzyme succinate dehydrogenase (SDH) (Ackermann et al., 1949), leading accordingly to succinate accumulation (Cordes T. et al., 2016). By inhibiting SDH, an enzyme critical for the inflammatory response (E. L. Mills et al., 2016), itaconate ameliorates inflammation in vitro and in vivo during macrophage activation and ischemia-reperfusion injury (Lampropoulou V. et al., 2016).

Like fumaric acid, itaconic acid is an α,β-unsaturated carboxylic acid. As such, it is a Michael acceptor which induces a global electrophilic stress response. In this regard, the itaconic acid diesterdimethyl itaconate (DM1), like DMF, produces an anti-inflammatory response, reducing the expression levels of pro-inflammatory cytokines IL-1β, IL-6, IL-12 and IL-18 in lipopolysaccharide (LPS)-stimulated bone marrow-derived macrophages (WO2017/142855A1, incorporated herein by reference). This response appears to be mediated, in part, by NRF2 activation, via alkylation of KEAP1 cysteine residues by the electrophilic α,β-unsaturated ester moiety (Mills E. L. et al., 2018), which enhances the expression of downstream genes with anti-oxidant and anti-inflammatory capacities. Nevertheless, not all of the pronounced immunoregulatory effects engendered by DMI can be attributed to NRF2 activation. In particular, the modulation of IκBζ by DMI is independent of NRF2 and is mediated via upregulation of ATF3, a global negative regulator of immune activation that downregulates various cytokines, such as IL-6 (Bambouskova M. et al., 2018). Moreover, by inhibiting IκBζ protein production, DMI ameliorates IL-17-mediated pathologies, highlighting the therapeutic potential of this regulatory pathway (WO2019/036509A1, incorporated herein by reference). Further highlighting its pharmacologic potential, DMI has recently been reported to 1) demonstrate a protective effect on cerebral ischemia/reperfusion injury, thereby offering potential for the treatment of ischemic stroke (Zhang D. et al., 2019); 2) provide protection from the cardiotoxic effects of doxorubicin (Shan Q. et al., 2019); and 3) protect against lippolysacchride-induced mastitis in mice by activating MAPKs and NRFrf2 while inhibiting NF-κB signaling pathways (Zhao C. et al., 2019). Furthermore, DMI is said to have utility in preventing and treating ulcerative colitis and canceration thereof (CN110731955, Sun Yat-sen University Cancer Center); and has been reported to protect against fungal keratitis by activating the NRF2/HO-1 signalling pathway (Gu L. et al., 2020). Nevertheless, it should be noted that DMI is not metabolised to itaconic acid intracellularly (ElAzzouny M. et al., 2017). Other α,β-unsaturated esters exhibit IL-1β-lowering effects in macrophages by inhibiting the NLRP3 inflammasome (Cocco M. et al., 2017 and 2014), and have been demonstrated to inhibit the TLR4 pathway, leading ultimately to suppression of LPS-induced stimulation of NF-κB, tumour necrosis factor (TNF)-α, IL-1β and nitric oxide release (Zhang S. et al., 2012).

Other itaconic acid derivatives have been demonstrated to elicit anti-inflammatory effects (Bagavant G. et al., 1994). A notable example is 4-octyl itaconic acid (4OI), an itaconate derivative with improved cellular uptake. Since the α,β-unsaturated carboxylic acid is not esterified in 4OI, this electrophile exhibits low reactivity with biological thiols (Schmidt T. J. et al., 2007), much like the situation encountered with itaconic acid itself. As a result of its low reactivity/electrophilicity, the NRF2-activating effects of 4OI are not attenuated by GSH, in contrast to the findings with the much more reactive DMI. In this latter case, the α,β-unsaturated carboxylic acid is esterified and, as a consequence, the IL-6-lowering and NRF2-activating effects of DMI are reversed by the thiols N-acetylcysteine and GSH, respectively. Through the reaction with KEAP1 and the resulting NRF2 activation, as well as GAPDH inhibition (Liao S.-T. et al., 2019), 4OI has been demonstrated to produce a wide range of interesting biological effects, including: 1) protection of neuronal cells from hydrogen peroxide (Liu H. et al., 2018); 2) inhibition of proinflammatory cytokine production in peripheral blood mononuclear cells of SLE patients (Tang C. et al., 2018); and 3) protection of human umbilical vein endothelial cells from high glucose (Tang C. et al., 2019); 4) inhibition of osteoclastogenesis by suppressing the E3 ubiquitin ligase Hrd1 and activating NRF2 signaling (Sun X. et al., 2019); 5) induction of repression of STING by NRF2 and type I IFN production in cells from patients with STING-dependent interferonopathies (Olagnier D. et al., 2018); 6) protection against renal fibrosis via inhibiting the TGF-beta/Smad pathway, autophagy and reducing generation of reactive oxygen species (Tian F. et al., 2020); 7) reduction of brain viral burden in mice intracranially injected with Zika virus (Daniels B. P. et al. 2019); and 8) protection against liver ischemia-reperfusion injury (Yi F. et al. 2020). Furthermore, itaconate has been reported to modulate tricarboxylic acid and redox metabolism to mitigate reperfusion injury (Cordes T. et al., 2020). In addition, raised plasma itaconate levels demonstrate a clear correlation with reduction in rheumatoid arthritis disease activity scores following commencement of therapy with conventional disease modifying anti-rheumatic drug (cDMARD) therapy (Daly R. et al. 2020).

Artyomov et al. (WO2017/142855; WO2019/036509) disclose the use of itaconate, malonate or a derivative thereof as an immunomodulatory agent. WO2020/222011, WO2020/222010, WO2021130492, WO2022/029438, WO2022/038365, WO2022/090714, WO2022/090723 and WO2022/090724 (Sitryx Therapeutics) disclose certain itaconate derivatives.

In spite of the above findings, there remains a need to identify and develop new therapeutics possessing enhanced properties compared to currently marketed anti-inflammatory agents, such as DMF. The present inventors have now discovered novel itaconate compounds which are more effective at reducing cytokine release in cells and/or in activating NRF2-driven effects than dimethyl itaconate. These properties, amongst others, including enhanced metabolic and hydrolytic stability, make them potentially more effective than dimethyl itaconate. Such compounds therefore possess excellent anti-inflammatory properties.

### SUMMARY OF THE INVENTION

The present invention provides a compound of formula (I):

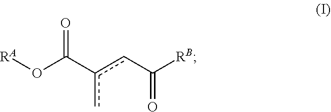

wherein the moiety:

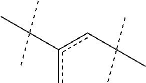

represents

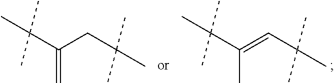

R^{A} is:

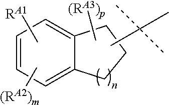

wherein:
R^{A1} is selected from the group consisting of H, halo, C₁₋₄ alkyl, C₁₋₄ haloalkyl, C₁₋₄ alkoxy, C₁₋₄ haloalkoxy, —SC₁₋₄ alkyl, —SC₁₋₄ haloalkyl and SF₅;
R^{A2} is selected from the group consisting of halo, C₁₋₄ alkyl, C₁₋₄ haloalkyl, C₁₋₄ alkoxy and C₁₋₄ haloalkoxy;
   R^{A3} is C₁₋₂ alkyl;
   m is 0, 1 or 2;
   n is 1 or 2; and
   p is 0, 1, 2, 3 or 4; or
R^{A} is:

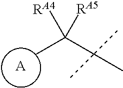

wherein:
   R^{A4} is H, C₁₋₄ alkyl, C₁₋₄ haloalkyl or C≡CH; and
   R^{A5} is H, C₁₋₄ alkyl or C₁₋₄ haloalkyl;
   or R^{A4} and R^{A5} join to form a C₃₋₆ cycloalkyl ring or a 4-6 membered heterocyclic ring wherein the C₃₋₆ cycloalkyl ring is optionally substituted by one or more R^{A6}, wherein R^{A6} is independently selected from the group consisting of fluoro, methyl and cyano, or two R^{A6} groups which are attached to the same carbon atom join to form a C₃₋₄ cycloalkyl ring;
   A is phenyl or 5-6-membered heteroaryl wherein when A is phenyl or 6-membered heteroaryl A is optionally substituted by one or more R^{A7}, and when A is 5-membered heteroaryl A is substituted by one or more R^{A7}, wherein when A is phenyl substituted by one or more R^{A7}, one R^{A7} is in the 4-position with respect to C(R^{A4})(R^{A5});
   R^{A7} is independently selected from the group consisting of halo, C₁₋₄ alkyl, C₁₋₄ haloalkyl, C₁₋₄ haloalkoxy, SF₅, SC₁₋₄ alkyl and SC₁₋₄ haloalkyl; and R^{B} is NR^{B1}R^{B2} or OR^{B3};
wherein:
   R^{B1} and R^{B2} are independently selected from the group consisting of H, C₁₋₄ alkyl, C₁₋₄ haloalkyl and 4-7-membered heterocyclyl wherein R^{B1} and/or R^{B2} are optionally substituted by one or more R^{B4} wherein R^{B4} is selected from the group consisting of C₁₋₂ alkyl, NH₂, N(C₁₋₂ alkyl)₂, hydroxy, oxo, 5-7-membered heterocyclyl and 5-6-membered heteroaryl optionally substituted by C₁₋₂ alkyl; and
   R^{B3} is selected from the group consisting of H, C₁₋₄ alkyl, C₁₋₄ haloalkyl, CH₂COOH, CH₂CH₂COOH, CH₂tetrazolyl and CH₂CH₂tetrazolyl, wherein R^{B3} is optionally substituted on an available carbon atom by one or more R^{B3′} wherein R^{B3′} is selected from the group consisting of difluoromethyl, trifluoromethyl and methyl; and/or wherein R^{B3} is optionally substituted by two R^{B3′} groups which are attached to the same carbon atom and join to form a C₃₋₆ cycloalkyl or a 4-6-membered heterocyclyl ring;
      or a pharmaceutically acceptable salt and/or solvate thereof.

The present invention provides a pharmaceutical composition comprising a compound of formula (I) or a pharmaceutically acceptable salt and/or solvate thereof.

The present invention provides a compound of formula (I) or a pharmaceutically acceptable salt and/or solvate thereof for use as a medicament.

The present invention provides a compound of formula (I) or a pharmaceutically acceptable salt and/or solvate thereof for use in treating or preventing an inflammatory disease or a disease associated with an undesirable immune response.

The present invention provides the use of a compound of formula (I) or a pharmaceutically acceptable salt and/or solvate thereof in the manufacture of a medicament for treating or preventing an inflammatory disease or a disease associated with an undesirable immune response.

The present invention provides a method of treating or preventing an inflammatory disease or a disease associated with an undesirable immune response, which comprises administering a compound of formula (I) or a pharmaceutically acceptable salt and/or solvate thereof.

Also provided are intermediate compounds of use in the preparation of compounds of formula (I).

### DETAILED DESCRIPTION OF THE INVENTION

### Compounds of Formula (I)

Embodiments and preferences set out herein with respect to the compound of formula (I) apply equally to the pharmaceutical composition, compound for use, use and method aspects of the invention.

As used herein, the term “C₁₋₄ alkyl” refers to a straight or branched fully saturated hydrocarbon group having the specified number of carbon atoms. The term encompasses methyl, ethyl, n-propyl, iso-propyl, n-butyl, iso-butyl, sec-butyl and tert-butyl. The term “alkyl” also encompasses “alkylene” which is a bifunctional straight or branched fully saturated hydrocarbon group having the stated number of carbon atoms. Example “alkylene” groups include methylene, ethylene, n-propylene and n-butylene.

The term “C₁₋₄ alkoxy” refers to an alkyl group, such as those defined above, singularly bonded via an oxygen atom. Examples of alkoxy groups include OCH₃.

The term “halo” refers to fluorine, chlorine, bromine or iodine. Particular examples of halo are fluorine, chlorine and bromine, especially fluorine.

The term “C₁₋₄ haloalkyl” refers to a straight or a branched fully saturated hydrocarbon chain containing the specified number of carbon atoms and at least one halogen atom, such as fluoro or chloro, especially fluoro. An example of haloalkyl is CF₃. Further examples of haloalkyl are CHF₂, CF₂CH₃ and CH₂CF₃.

The term “C₁₋₄ haloalkoxy” refers to a haloalkyl group as defined above, singularly bonded via an oxygen atom. Examples of haloalkoxy groups include OCF₃, OCHF₂ and OCH₂CF₃.

The term “C₃₋₆ cycloalkyl” such as “C₃₋₄ cycloalkyl” refers to a fully saturated cyclic hydrocarbon group having the specified number of carbon atoms. The term encompasses cyclopropyl, cyclobutyl, cyclopentyl and cyclohexyl.

The term “4-7-membered heterocyclic ring” (such as “4-6-membered heterocyclic ring” or “5-7-membered heterocyclic ring”) refers to a non-aromatic cyclic group having the stated number of ring atoms and wherein at least one of the ring atoms is a heteroatom selected from N, O, S and B. The term “heterocyclic ring” is interchangeable with “heterocyclyl”. The term encompasses azetidinyl, oxetanyl, thietanyl, pyrrolidinyl, tetrahydrofuranyl, tetrahydrothienyl, tetrahydropyranyl, piperidinyl, piperazinyl, morpholinyl, thiomorpholinyl, oxepanyl, thiepanyl and azepanyl. 4-7-membered heterocyclyl groups can typically be substituted by one or more (e.g. one or two) oxo groups. Suitably, a sulphur atom is substituted by one or two oxo groups thus forming S═O or SO₂.

The term “5-6-membered heteroaryl” refers to a cyclic group with aromatic character wherein at least one of the atoms in the cyclic group is a heteroatom independently selected from N, O and S. The term encompasses pyrrolyl, furanyl, thienyl, imidazolyl, pyrazolyl, thiazolyl, oxadiazolyl, thiadiazolyl, triazolyl, oxazolyl, tetrazolyl, pyridyl, pyrimidinyl, pyradizinyl and pyrazinyl.

The term “tetrazolyl” refers to a 5-(1H-tetrazolyl) substituent where the tetrazole is linked to the rest of the molecule via a carbon atom:

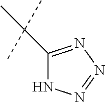

wherein the dashed line indicates the point of attachment to the rest of the molecule.

Where substituents are indicated as being optionally substituted in formula (I) in the embodiments and preferences set out below, the optional substituent may be attached to an available carbon atom, which means a carbon atom which is attached to a hydrogen atom i.e. a C—H group. The optional substituent replaces the hydrogen atom attached to the carbon atom.

In one embodiment, the moiety:

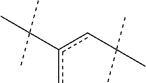

represents

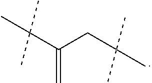

The carbon-carbon double bond in this structure is referred to as “exo”.

In another embodiment, the moiety:

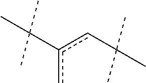

represents

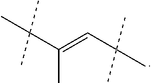

The carbon-carbon double bond in this structure is referred to as “endo”.

In the endo embodiment, the double bond may be cis or trans such that both of the following moieties are covered:

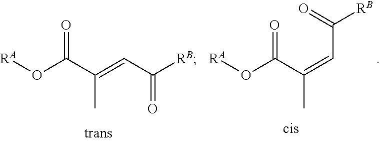

Suitably, the endo double bond in the compound of formula (I) is trans.

The compounds of formula (I) in which the carbon-carbon double bond is endo can generally be obtained by isomerisation from compounds of formula (I) in which the carbon-carbon double bond is exo and such isomerisation may occur in in vitro assays or in vivo following administration of the exo compound. In some cases, isomerisation in in vitro assays, such as in vitro hepatocyte stability assays, or in vivo following administration of the exo compound may be partial and thus lead to a mixture of the endo and exo compounds resulting. In some cases, the mixture of endo and exo isomers may contribute to the activity observed in a particular assay. Suitably, compounds of formula (I), such as those in which the carbon-carbon double bond is exo, are stable to isomerisation.

In one embodiment, R^{A} is:

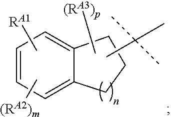

wherein R^{A1}, R^{A2}, R^{A3}, m, n and p are defined above.

In one embodiment, R^{A1} is C₁₋₄ haloalkyl such as CF₃.

In one embodiment, m is 0.

In one embodiment, n is 1.

In one embodiment, p is 0.

In another embodiment, R^{A} is:

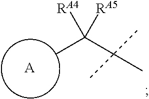

wherein A, R^{A4} and R^{A5} are defined above.

In one embodiment, R^{A4} is C₁₋₄ alkyl such as methyl.

In one embodiment, R^{A5} is H.

Suitably, when R^{A4} and R^{A5} are different (e.g. when R^{A4} is H and R^{A5} is methyl), the carbon atom to which R^{A4} and R^{A5} are attached has the following stereochemistry:

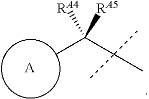

In one embodiment, R^{A4} and R^{A5} join to form a C₃₋₆ cycloalkyl ring such as a C₄ cycloalkyl ring.

In one embodiment, the C₃₋₆ cycloalkyl ring is not substituted. In one embodiment, the C₃₋₆ cycloalkyl ring is substituted by one or more (such as one, two or three e.g. one) R^{A6}, wherein R^{A6} is independently selected from the group consisting of fluoro, methyl and cyano, or two R^{A6} groups which are attached to the same carbon atom join to form a C₃₋₄ cycloalkyl ring.

In one embodiment, R^{A4} and R^{A5} join to form a 4-6 membered heterocyclic ring such as a 4-membered heterocyclic ring e.g., oxetanyl.

In one embodiment, A is phenyl. In a second embodiment, A is 5-6-membered heteroaryl, such as thienyl or pyridinyl. Suitably, A is 5-membered heteroaryl. Alternatively, A is 6-membered heteroaryl.

In one embodiment, and when A is phenyl or 6-membered heteroaryl, A is not substituted. In another embodiment, A is phenyl or 5-6-membered heteroaryl and is substituted by one or more (such as one, two or three e.g. one) R^{A7}, wherein R^{A7} is defined above.

In one embodiment, R^{A7} is halo such as F. In a second embodiment, R^{A7} is C₁₋₄ haloalkyl such as CF₃.

When A is phenyl substituted with one or more R^{A7}, one R^{A7} is in the 4-position with respect to C(R^{A4})(R^{A5}):

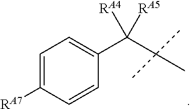

In this embodiment, suitably, R^{A7} is C₁₋₄ haloalkyl such as CF₃.

In one embodiment, R^{B} is NR^{B1}R^{B2}. In a second embodiment, R^{B} is OR^{B3}.

In one embodiment, R^{B1} is H. In a second embodiment, R^{B1} is C₁₋₄ alkyl such as methyl. In a third embodiment, R^{B1} is C₁₋₄ haloalkyl. In a fourth embodiment, R^{B1} is 4-7-membered heterocyclyl such as tetrahydropyranyl or optionally substituted thietanyl, for example dioxidothietanyl. In some cases, R^{B1} is 5-7-membered heterocyclyl such as tetrahydropyranyl.

In one embodiment, R^{B2} is H. In a second embodiment, R^{B2} is C₁₋₄ alkyl such as methyl. In a third embodiment, R^{B2} is C₁₋₄ haloalkyl. In a fourth embodiment, R^{B2} is 4-7-membered heterocyclyl such as tetrahydropyranyl or optionally substituted thietanyl, for example dioxidothietanyl. In some cases, R^{B2} is 5-7-membered heterocyclyl such as tetrahydropyranyl.

Suitably, both R^{B1} and R^{B2} are H, or both R^{B1} and R^{B2} are methyl. Alternatively, R^{B1} is H and R^{B2} is methyl.

Suitably, at least one of R^{B1} and R^{B2} is H.

In one embodiment, R^{B1} and/or R^{B2} are not substituted. In another embodiment, R^{B1} and/or R^{B2} are substituted by one or more (such as one, two or three, e.g., one) R^{B4} wherein R^{B4} is selected from the group consisting of C₁₋₂ alkyl, NH₂, N(C₁₋₂ alkyl)₂, hydroxy, oxo, 5-7-membered heterocyclyl and 5-6-membered heteroaryl optionally substituted by C₁₋₂ alkyl.

In one embodiment, R^{B4} is C₁₋₂ alkyl. In a second embodiment, R^{B4} is NH₂. In a third embodiment, R^{B4} is N(C₁₋₂ alkyl)₂. In a fourth embodiment, R^{B4} is hydroxy. In a fifth embodiment, R^{B4} is oxo (═O). In a sixth embodiment, R^{B4} is 5-7-membered heterocyclyl. In a seventh embodiment, R^{B4} is 5-6-membered heteroaryl optionally substituted by C₁₋₂ alkyl.

In one embodiment R^{B} is OR^{B3}.

In one embodiment, R^{B3} is H. In a second embodiment, R^{B3} is C₁₋₄ alkyl such as methyl. In a third embodiment, R^{B3} is CH₂COOH.

In one embodiment, R^{B3} is not substituted. In another embodiment, R^{B3} is optionally substituted on an available carbon atom by one or more (such as one, two or three e.g. one) R^{B3′} wherein R^{B3′} is selected from the group consisting of difluoromethyl, trifluoromethyl and methyl; and/or wherein R^{B3} is optionally substituted by two R^{B3′} groups which are attached to the same carbon atom and join to form a C₃₋₆ cycloalkyl or a 4-6-membered heterocyclyl ring.

The term “available carbon atom” means any carbon atom which forms a C—H bond. The substituent replaces the hydrogen atom attached to the carbon atom. The skilled person will appreciate that when R^{B3} is H, there is no “available carbon atom” for substitution.

In one embodiment, the molecular weight of the compound of formula (I) is 150 Da-450 Da, suitably 200 Da-400 Da.

In one embodiment there is provided a compound of formula (I), selected from the group consisting of:
(S)-3-((1-(4-(trifluoromethyl)phenyl)ethoxy)carbonyl)but-3-enoic acid;
3-((1-(4-(trifluoromethyl)phenyl)cyclobutoxy)carbonyl)but-3-enoic acid;
3-((1-(3-fluoro-4-(trifluoromethyl)phenyl)cyclobutoxy)carbonyl)but-3-enoic acid;
3-(((3-(4-(trifluoromethyl)phenyl)oxetane-3-yl)oxy)carbonyl)but-3-enoic acid;
3-((1-(5-(trifluoromethyl)pyridin-2-yl)cyclobutoxy)carbonyl)but-3-enoic acid;
3-((1-(5-(trifluoromethyl)thiophen-2-yl)cyclobutoxy)carbonyl)but-3-enoic acid;
3-(((6-(trifluoromethyl)-1,2,3,4-tetrahydronaphthalen-1-yl)oxy)carbonyl)but-3-enoic acid;
4-methyl 1-(1-(4-(trifluoromethyl)phenyl)cyclobutyl) 2-methylenesuccinate;
1-(4-(trifluoromethyl)phenyl)cyclobutyl 4-(methylamino)-2-methylene-4-oxobutanoate;
1-(4-(trifluoromethyl)phenyl)cyclobutyl 4-(dimethylamino)-2-methylene-4-oxobutanoate;
1-(4-(trifluoromethyl)phenyl)cyclobutyl 4-amino-2-methylene-4-oxobutanoate;
1-(4-(trifluoromethyl)phenyl)cyclobutyl 4-((2-(dimethylamino)ethyl)amino)-2-methylene-4-oxobutanoate;
1-(4-(trifluoromethyl)phenyl)cyclobutyl 2-methylene-4-((2-morpholinoethyl)amino)-4-oxobutanoate;
1-(4-(trifluoromethyl)phenyl)cyclobutyl 2-methylene-4-((1-methylpiperidin-4-yl)amino)-4-oxobutanoate;
1-(4-(trifluoromethyl)phenyl)cyclobutyl 4-(((1H-imidazol-2-yl)methyl)amino)-2-methylene-4-oxobutanoate;
1-(4-(trifluoromethyl)phenyl)cyclobutyl 4-(((1-methyl-1H-imidazol-2-yl)methyl)amino)-2-methylene-4-oxobutanoate;
1-(4-(trifluoromethyl)phenyl)cyclobutyl 4-((1-amino-3-hydroxy-1-oxopropan-2-yl)amino)-2-methylene-4-oxobutanoate;
1-(4-(trifluoromethyl)phenyl)cyclobutyl 4-((1,1-dioxidothietan-3-yl)amino)-2-methylene-4-oxobutanoate;
1-(4-(trifluoromethyl)phenyl)cyclobutyl 4-((2-hydroxyethyl)amino)-2-methylene-4-oxobutanoate;
1-(4-(trifluoromethyl)phenyl)cyclobutyl 2-methylene-4-oxo-4-((tetrahydro-2H-pyran-4-yl)amino)butanoate;
1-(4-(trifluoromethyl)phenyl)cyclobutyl 4-((2-amino-2-oxoethyl)amino)-2-methylene-4-oxobutanoate;
2-((3-((1-(4-(trifluoromethyl)phenyl)cyclobutoxy)carbonyl)but-3-enoyl)oxy)acetic acid; and
3-methyl-4-oxo-4-(1-(4-(trifluoromethyl)phenyl)cyclobutoxy)but-2-enoic acid;
   or a pharmaceutically acceptable salt and/or solvate of any one thereof.

Compounds of formula (I) may be prepared as set out in the Examples and as set out in the following schemes.

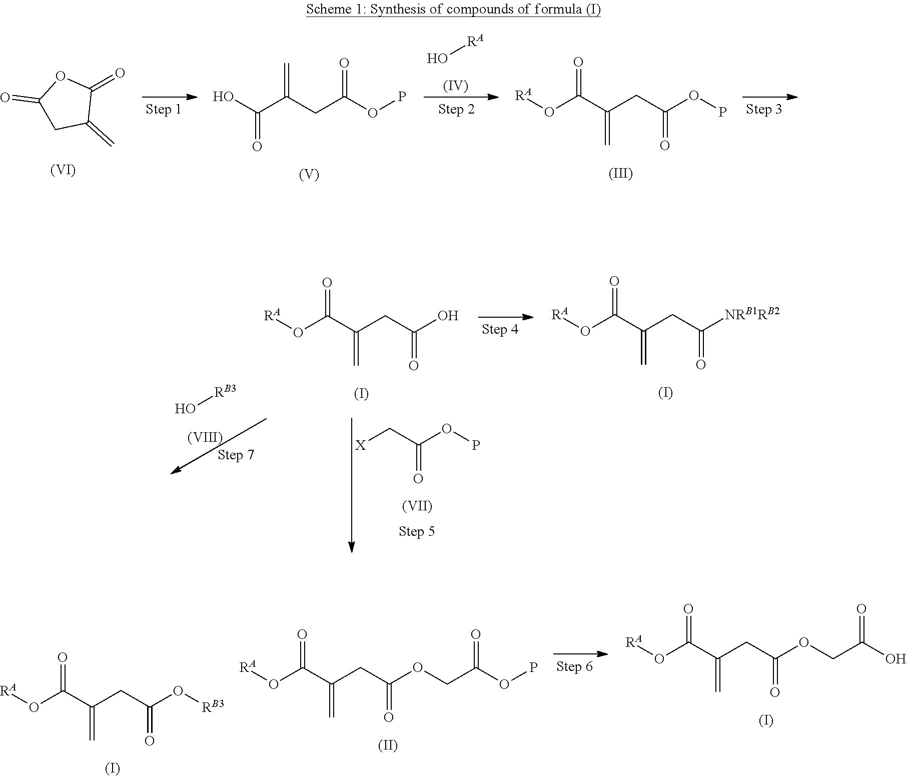

wherein R^{A}, R^{B1}, R^{B2} and R^{B3} are defined elsewhere herein, P is a carboxylic acid protecting group such as CH₂CCl₃, and X is a leaving group such as halo, e.g., bromo.

Step 1: Lewis acid catalysed ring opening of itaconic anhydride (VI) using HO—P provides α,β-unsaturated carboxylic acids (V).

Step 2: Esterification of carboxylic acids (V) with alcohols (IV) under standard conditions (such as DCC, DMAP in DCM) provides α,β-unsaturated esters of formula (III). Alcohols (IV) are commercially available or can be made according to the Examples.

Step 3: Protecting group P is removed under conditions known to the person skilled in the art to give compounds of formula (I) wherein R^{B3} is H. For example, when P is CH₂CCl₃, Zn/NH₄OAc may be used to remove this protecting group.

Step 4: Carboxylic acids of formula (I), wherein R^{B3} is H, may be converted to compounds of formula (I), wherein R^{B} is NR^{B1}R^{B2}, using standard amide coupling conditions, such as using T3P, a base, such as Et₃N, in a solvent, such as EtOAc, or using HATU in the presence of a base, such as TEA, in a solvent such as dimethylformamide.

Step 5: Compounds of formula (II) may be obtained by reacting compounds of formula (I), wherein R^{B3} is H, with a protected carboxylic acid derivative (VII).

Step 6: Removal of protecting group P under conditions known to the person skilled in the art provides compounds of formula (I) wherein R^{B3} is CH₂COOH. For example, when P is CH₂CCl₃, Zn/NH₄OAc may be used to remove this protecting group.

Step 7: Carboxylic acids of formula (I), wherein R^{B3} is H, may be converted to compounds of formula (I), wherein R^{B} is OR^{B3}, by esterification of carboxylic acids of formula (I), for example, by using HO—R^{B3} of formula (VIII). In this step, the protected derivative of compounds of formula (VIII), HO—R^{B3P} of formula (IX), may also be used. For example, when R^{B3} is —CH₂CH₂COOH, the compound of formula (I) wherein R^{B} is OH may be coupled with an ester of the form HOCH₂CH₂CO₂P, wherein P is a carboxylic acid protecting group as defined herein, followed by removal of the protecting group.

Compounds of formula (I) wherein

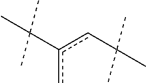

represents

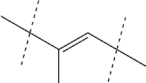

may be obtained by isomerisation of compounds of formula (I) wherein

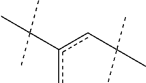

represents

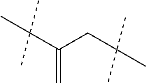

under basic conditions, such as those described in the Examples.

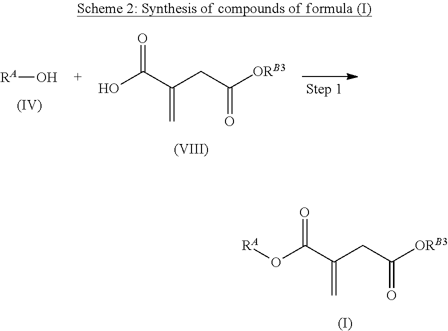

wherein R^{A} and R^{B3} are defined elsewhere herein.

Step 1: Esterification of commercially available carboxylic acids (VIII) with alcohols of formula (IV) using standard coupling conditions such as DCC and DMAP in a solvent such as DCM, provides compounds of formula (I).

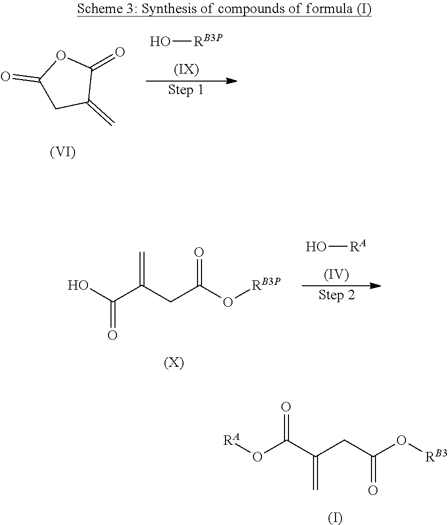

wherein R^{A} and R^{B3} are defined elsewhere herein, and R^{B3P} is a protected derivative of R^{B3}. For example, when R^{B3} comprises a carboxylic acid, the acid group may be protected with a carboxylic acid protecting group such as C₁₋₆ alkyl, e.g., tert-butyl, or para-methoxybenzyl, and when R^{B3} comprises a tetrazolyl protecting group, the tetrazolyl group may be protected using a tetrazolyl protecting group such as para-methoxybenzyl or trityl.

Step 1: Lewis acid catalysed ring opening of itaconic anhydride using alcohols of formula (IX) provides α,β-unsaturated carboxylic acids (X).

Step 2: Esterification of carboxylic acids (X) with alcohols (IV) under standard conditions (such as DCC, DMAP in DCM) provides compounds of formula (I).

When R^{B} comprises CH₂COOH or CH₂CH₂COOH, suitably the carboxylic acid protecting group replaces the hydrogen atom attached to an oxygen atom, i.e., “—R^{B3P}” is CH₂COO—P or CH₂CH₂COO—P, wherein P is the protecting group

When R^{B} comprises CH₂tetrazolyl or CH₂CH₂tetrazolyl, suitably the tetrazolyl protecting group replaces the hydrogen atom attached to a nitrogen atom:

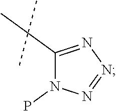

### i.e., “—R^{B3P}” is

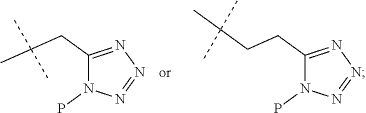

wherein the dashed line indicates attachment to the remainder of the compound of formula (I), and P is the protecting group.

If no R^{B3} protecting group is required, for example, when R^{B3} is C₁₋₄ alkyl or C₁₋₄ haloalkyl, the synthetic route in Scheme 3 may be followed, using a compound of formula HO—R^{B3} (VIII) in step 1, instead of HO-R^{B3P}

Thus, in one embodiment, there is provided a compound of formula (II):

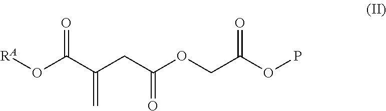

wherein R^{A} is defined elsewhere herein and P is a carboxylic acid protecting group such as CH₂CCl₃;
or salts thereof.

In one embodiment, there is provided a compound of formula (III):

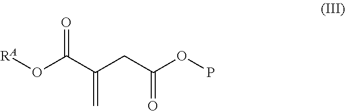

wherein R^{A} is defined elsewhere herein and P is a carboxylic acid protecting group such as CH₂CCl₃;
or salts thereof.

In one embodiment, there is provided a compound of formula (X):

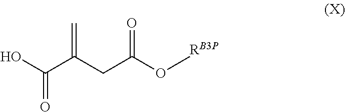

wherein R^{B3P} is defined elsewhere herein;
or salts thereof.

In one embodiment, there is provided a process for the preparation of compounds of formula (I), or salts, such as pharmaceutically acceptable salts, thereof, which comprises the step of deprotecting compounds of formula (III):

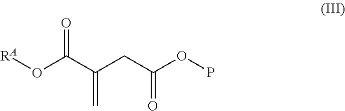

or salts thereof;
wherein R^{A} is defined elsewhere herein and P is a carboxylic acid protecting group such as CH₂CCl₃.

It will be appreciated that for use in therapy the salts of the compounds of formula (I) should be pharmaceutically acceptable. Suitable pharmaceutically acceptable salts will be apparent to those skilled in the art. Pharmaceutically acceptable salts include acid addition salts, suitably salts of compounds of the invention comprising a basic group such as an amino group, formed with inorganic acids, e.g., hydrochloric acid, hydrobromic acid, sulfuric acid, nitric acid or phosphoric acid. Also included are salts formed with organic acids e.g. succinic acid, maleic acid, acetic acid, fumaric acid, citric acid, tartaric acid, benzoic acid, p-toluenesulfonic acid, methanesulfonic acid, naphthalenesulfonic acid and 1,5-naphthalenedisulfonic acid. Other salts, e.g., oxalates or formates, may be used, for example in the isolation of compounds of formula (I) and are included within the scope of this invention, as are basic addition salts such as sodium, potassium, calcium, aluminium, zinc, magnesium and other metal salts.

Pharmaceutically acceptable salts may also be formed with organic bases such as basic amines e.g. with ammonia, meglumine, tromethamine, piperazine, arginine, choline, diethylamine, benzathine or lysine. Thus, in one embodiment there is provided a compound of formula (I) in the form of a pharmaceutically acceptable salt. Alternatively, there is provided a compound of formula (I) in the form of a free acid. When the compound contains a basic group as well as the free acid it may be Zwitterionic.

Suitably, the compound of formula (I) is not a salt e.g. is not a pharmaceutically acceptable salt.

Suitably, where the compound of formula (I) is in the form of a salt, the pharmaceutically acceptable salt is a basic addition salt such as a carboxylate salt formed with a group 1 metal (e.g. a sodium or potassium salt), a group 2 metal (e.g. a magnesium or calcium salt) or an ammonium salt of a basic amine (e.g. an NH_{4′} salt), such as a sodium salt.

The compounds of formula (I) may be prepared in crystalline or non-crystalline form and, if crystalline, may optionally be solvated, e.g. as the hydrate. This invention includes within its scope stoichiometric solvates (e.g. hydrates) as well as compounds containing variable amounts of solvent (e.g. water). Suitably, the compound of formula (I) is not a solvate.

The invention extends to a pharmaceutically acceptable derivative thereof, such as a pharmaceutically acceptable prodrug of compounds of formula (I). Typical prodrugs of compounds of formula (I) which comprise a carboxylic acid include ester (e.g. C₁₋₆ alkyl e.g. C₁₋₄ alkyl ester) derivatives thereof. Thus, in one embodiment, the compound of formula (I) is provided as a pharmaceutically acceptable prodrug. In another embodiment, the compound of formula (I) is not provided as a pharmaceutically acceptable prodrug.

It is to be understood that the present invention encompasses all isomers of compounds of formula (I) including all geometric, tautomeric and optical forms, and mixtures thereof (e.g. racemic mixtures). Where additional chiral centres are present in compounds of formula (I), the present invention includes within its scope all possible diastereoisomers, including mixtures thereof. The different isomeric forms may be separated or resolved one from the other by conventional methods, or any given isomer may be obtained by conventional synthetic methods or by stereospecific or asymmetric syntheses.

The present invention also includes all isotopic forms of the compounds provided herein, whether in a form (i) wherein all atoms of a given atomic number have a mass number (or mixture of mass numbers) which predominates in nature (referred to herein as the “natural isotopic form”) or (ii) wherein one or more atoms are replaced by atoms having the same atomic number, but a mass number different from the mass number of atoms which predominates in nature (referred to herein as an “unnatural variant isotopic form”). It is understood that an atom may naturally exist as a mixture of mass numbers. The term “unnatural variant isotopic form” also includes embodiments in which the proportion of an atom of given atomic number having a mass number found less commonly in nature (referred to herein as an “uncommon isotope”) has been increased relative to that which is naturally occurring e.g. to the level of >20%, >50%, >75%, >90%, >95% or >99% by number of the atoms of that atomic number (the latter embodiment referred to as an “isotopically enriched variant form”). The term “unnatural variant isotopic form” also includes embodiments in which the proportion of an uncommon isotope has been reduced relative to that which is naturally occurring. Isotopic forms may include radioactive forms (i.e. they incorporate radioisotopes) and non-radioactive forms. Radioactive forms will typically be isotopically enriched variant forms.

An unnatural variant isotopic form of a compound may thus contain one or more artificial or uncommon isotopes such as deuterium (²H or D), carbon-11 (¹¹C), carbon-13 (¹³C), carbon-14 (¹⁴C), nitrogen-13 (¹³N), nitrogen-15 (¹⁵N), oxygen-15 (¹⁵O), oxygen-17 (¹⁷O), oxygen-18 (¹⁸O), phosphorus-32 (³²P), sulphur-35 (³⁵S), chlorine-36 (³⁶Cl), chlorine-37 (³⁷Cl), fluorine-18 (¹⁸F) iodine-123 (¹²³I), iodine-125 (¹²⁵I) in one or more atoms or may contain an increased proportion of said isotopes as compared with the proportion that predominates in nature in one or more atoms.

Unnatural variant isotopic forms comprising radioisotopes may, for example, be used for drug and/or substrate tissue distribution studies. The radioactive isotopes tritium, i.e. ³H, and carbon-14, i.e. ¹⁴C, are particularly useful for this purpose in view of their ease of incorporation and ready means of detection. Unnatural variant isotopic forms which incorporate deuterium i.e. ²H or D may afford certain therapeutic advantages resulting from greater metabolic stability, for example, increased in vivo half-life or reduced dosage requirements, and hence may be preferred in some circumstances. Further, unnatural variant isotopic forms may be prepared which incorporate positron emitting isotopes, such as ¹¹C, ¹⁸F, ¹⁵O and ¹³N, and would be useful in positron emission topography (PET) studies for examining substrate receptor occupancy.

In one embodiment, the compounds of formula (I) are provided in a natural isotopic form. In one embodiment, the compounds of formula (I) are provided in an unnatural variant isotopic form. In a specific embodiment, the unnatural variant isotopic form is a form in which deuterium (i.e. ²H or D) is incorporated where hydrogen is specified in the chemical structure in one or more atoms of a compound of formula (I). In one embodiment, the atoms of the compounds of formula (I) are in an isotopic form which is not radioactive. In one embodiment, one or more atoms of the compounds of formula (I) are in an isotopic form which is radioactive. Suitably radioactive isotopes are stable isotopes. Suitably the unnatural variant isotopic form is a pharmaceutically acceptable form.

In one embodiment, a compound of formula (I) is provided whereby a single atom of the compound exists in an unnatural variant isotopic form. In another embodiment, a compound of formula (I) is provided whereby two or more atoms exist in an unnatural variant isotopic form.

Unnatural isotopic variant forms can generally be prepared by conventional techniques known to those skilled in the art or by processes described herein, e.g., processes analogous to those described in the accompanying Examples for preparing natural isotopic forms. Thus, unnatural isotopic variant forms could be prepared by using appropriate isotopically variant (or labelled) reagents in place of the normal reagents employed in the Examples. Since the compounds of formula (I) are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

### Therapeutic Indications

Compounds of formula (I) are of use in therapy, particularly for treating or preventing an inflammatory disease or a disease associated with an undesirable immune response. As shown in Biological Example 1 below, example compounds of formula (I) reduced cytokine release more effectively than dimethyl itaconate and 1-monomethyl itaconate as demonstrated by lower IC₅₀ values. Cytokines are important mediators of inflammation and immune-mediated disease as evidenced by the therapeutic benefit delivered by antibodies targeting them.

Thus, in a first aspect, the present invention provides a compound of formula (I) or a pharmaceutically acceptable salt and/or solvate thereof as defined herein, for use as a medicament. Also provided is a pharmaceutical composition comprising a compound of formula (I) or a pharmaceutically acceptable salt and/or solvate thereof as defined herein. Such a pharmaceutical composition contains the compound of formula (I) and a pharmaceutically acceptable carrier or excipient.

In a further aspect, the present invention provides a compound of formula (I) or a pharmaceutically acceptable salt and/or solvate thereof as defined herein, for use in treating or preventing an inflammatory disease or a disease associated with an undesirable immune response. In a further aspect, the present invention provides the use of a compound of formula (I) or a pharmaceutically acceptable salt and/or solvate thereof as defined herein, in the manufacture of a medicament for treating or preventing an inflammatory disease or a disease associated with an undesirable immune response. In a further aspect, the present invention provides a method of treating or preventing an inflammatory disease or a disease associated with an undesirable immune response, which comprises administering a compound of formula (I) or a pharmaceutically acceptable salt and/or solvate thereof as defined herein.

For all aspects of the invention, suitably the compound is administered to a subject in need thereof, wherein the subject is suitably a human subject.

In one embodiment is provided a compound of formula (I) or a pharmaceutically acceptable salt and/or solvate thereof as defined herein, for use in treating an inflammatory disease or disease associated with an undesirable immune response. In one embodiment of the invention is provided the use of a compound of formula (I) or a pharmaceutically acceptable salt and/or solvate thereof as defined herein, in the manufacture of a medicament for treating an inflammatory disease or a disease associated with an undesirable immune response. In one embodiment of the invention is provided a method of treating an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in preventing an inflammatory disease or a disease associated with an undesirable immune response. In one embodiment of the invention is provided the use of a compound of formula (I) or a pharmaceutically acceptable salt and/or solvate thereof as defined herein, in the manufacture of a medicament for preventing an inflammatory disease or a disease associated with an undesirable immune response. In one embodiment of the invention is provided a method of preventing an inflammatory disease or a disease associated with an undesirable immune response, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing an inflammatory disease. In one embodiment of the invention is provided the use of a compound of formula (I) or a pharmaceutically acceptable salt and/or solvate thereof as defined herein, in the manufacture of a medicament for treating or preventing an inflammatory disease. In one embodiment of the invention is provided a method of treating or preventing an inflammatory disease, which comprises administering a compound of formula (I) or a pharmaceutically acceptable salt and/or solvate thereof as defined herein.

In one embodiment is provided a compound of formula (I) or a pharmaceutically acceptable salt and/or solvate thereof as defined herein, for use in treating or preventing a disease associated with an undesirable immune response. In one embodiment of the invention is provided the use of a compound of formula (I) or a pharmaceutically acceptable salt and/or solvate thereof as defined herein, in the manufacture of a medicament for treating or preventing a disease associated with an undesirable immune response. In one embodiment of the invention is provided a method of treating or preventing a disease associated with an undesirable immune response, which comprises administering a compound of formula (I) or a pharmaceutically acceptable salt and/or solvate thereof as defined herein.

An undesirable immune response will typically be an immune response which gives rise to a pathology i.e. is a pathological immune response or reaction.

In one embodiment, the inflammatory disease or disease associated with an undesirable immune response is an auto-immune disease.

In one embodiment, the inflammatory disease or disease associated with an undesirable immune response is, or is associated with, a disease selected from the group consisting of: psoriasis (including chronic plaque, erythrodermic, pustular, guttate, inverse and nail variants), asthma, chronic obstructive pulmonary disease (COPD, including chronic bronchitis and emphysema), heart failure (including left ventricular failure), myocardial infarction, angina pectoris, other atherosclerosis and/or atherothrombosis-related disorders (including peripheral vascular disease and ischaemic stroke), a mitochondrial and neurodegenerative disease (such as Parkinson's disease, Alzheimer's disease, Huntington's disease, amyotrophic lateral sclerosis, retinitis pigmentosa or mitochondrial encephalomyopathy), autoimmune paraneoplastic retinopathy, transplantation rejection (including antibody-mediated and T cell-mediated forms), multiple sclerosis, transverse myelitis, ischaemia-reperfusion injury (e.g. during elective surgery such as cardiopulmonary bypass for coronary artery bypass grafting or other cardiac surgery, following percutaneous coronary intervention, following treatment of acute ST-elevation myocardial infarction or ischaemic stroke, organ transplantation, or acute compartment syndrome), AGE-induced genome damage, an inflammatory bowel disease (e.g. Crohn's disease or ulcerative colitis), primary sclerosing cholangitis (PSC), PSC-autoimmune hepatitis overlap syndrome, non-alcoholic fatty liver disease (non-alcoholic steatohepatitis), rheumatica, granuloma annulare, cutaneous lupus erythematosus (CLE), systemic lupus erythematosus (SLE), lupus nephritis, drug-induced lupus, autoimmune myocarditis or myopericarditis, Dressler's syndrome, giant cell myocarditis, post-pericardiotomy syndrome, drug-induced hypersensitivity syndromes (including hypersensitivity myocarditis), eczema, sarcoidosis, erythema nodosum, acute disseminated encephalomyelitis (ADEM), neuromyelitis optica spectrum disorders, MOG (myelin oligodendrocyte glycoprotein) antibody-associated disorders (including MOG-EM), optic neuritis, CLIPPERS (chronic lymphocytic inflammation with pontine perivascular enhancement responsive to steroids), diffuse myelinoclastic sclerosis, Addison's disease, alopecia areata, ankylosing spondylitis, other spondyloarthritides (including peripheral spondyloarthritis, that is associated with psoriasis, inflammatory bowel disease, reactive arthritis or juvenile onset forms), antiphospholipid antibody syndrome, autoimmune hemolytic anaemia, autoimmune hepatitis, autoimmune inner ear disease, pemphigoid (including bullous pemphigoid, mucous membrane pemphigoid, cicatricial pemphigoid, herpes gestationis or pemphigoid gestationis, ocular cicatricial pemphigoid), linear IgA disease, Behget's disease, celiac disease, Chagas disease, dermatomyositis, diabetes mellitus type I, endometriosis, Goodpasture's syndrome, Graves' disease, Guillain-Barre syndrome and its subtypes (including acute inflammatory demyelinating polyneuropathy, AIDP, acute motor axonal neuropathy (AMAN), acute motor and sensory axonal neuropathy (AMSAN), pharyngeal-cervical-brachial variant, Miller-Fisher variant and Bickerstaff's brainstem encephalitis), progressive inflammatory neuropathy, Hashimoto's disease, hidradenitis suppurativa, inclusion body myositis, necrotising myopathy, Kawasaki disease, IgA nephropathy, Henoch-Schonlein purpura, idiopathic thrombocytopenic purpura, thrombotic thrombocytopenic purpura (TTP), Evans' syndrome, interstitial cystitis, mixed connective tissue disease, undifferentiated connective tissue disease, morphea, myasthenia gravis (including MuSK antibody positive and seronegative variants), narcolepsy, neuromyotonia, pemphigus vulgaris, pernicious anaemia, psoriatic arthritis, polymyositis, primary biliary cholangitis (also known as primary biliary cirrhosis), rheumatoid arthritis, palindromic rheumatism, schizophrenia, autoimmune (meningo-)encephalitis syndromes, scleroderma, Sjogren's syndrome, stiff person syndrome, polymylagia rheumatica, giant cell arteritis (temporal arteritis), Takayasu arteritis, polyarteritis nodosa, Kawasaki disease, granulomatosis with polyangitis (GPA; formerly known as Wegener's granulomatosis), eosinophilic granulomatosis with polyangiitis (EGPA; formerly known as Churg-Strauss syndrome), microscopic polyarteritis/polyangiitis, hypocomplementaemic urticarial vasculitis, hypersensitivity vasculitis, cryoglobulinemia, thromboangiitis obliterans (Buerger's disease), vasculitis, leukocytoclastic vasculitis, vitiligo, acute disseminated encephalomyelitis, adrenoleukodystrophy, Alexander's disease, Alper's disease, balo concentric sclerosis or Marburg disease, cryptogenic organising pneumonia (formerly known as bronchiolitis obliterans organizing pneumonia), Canavan disease, central nervous system vasculitic syndrome, Charcot-Marie-Tooth disease, childhood ataxia with central nervous system hypomyelination, chronic inflammatory demyelinating polyneuropathy (CIDP), diabetic retinopathy, globoid cell leukodystrophy (Krabbe disease), graft-versus-host disease (GVHD) (including acute and chronic forms, as well as intestinal GVHD), hepatitis C (HCV) infection or complication, herpes simplex viral infection or complication, human immunodeficiency virus (HIV) infection or complication, lichen planus, monomelic amyotrophy, cystic fibrosis, pulmonary arterial hypertension (PAH, including idiopathic PAH), lung sarcoidosis, idiopathic pulmonary fibrosis, paediatric asthma, atopic dermatitis, allergic dermatitis, contact dermatitis, allergic rhinitis, rhinitis, sinusitis, conjunctivitis, allergic conjunctivitis, keratoconjunctivitis sicca, dry eye, xerophthalmia, glaucoma, macular oedema, diabetic macular oedema, central retinal vein occlusion (CRVO), macular degeneration (including dry and/or wet age related macular degeneration, AMD), post-operative cataract inflammation, uveitis (including posterior, anterior, intermediate and pan uveitis), iridocyclitis, scleritis, corneal graft and limbal cell transplant rejection, gluten sensitive enteropathy (coeliac disease), dermatitis herpetiformis, eosinophilic esophagitis, achalasia, autoimmune dysautonomia, autoimmune encephalomyelitis, autoimmune oophoritis, autoimmune orchitis, autoimmune pancreatitis, aortitis and periaortitis, autoimmune retinopathy, autoimmune urticaria, (idiopathic) Castleman's disease, Cogan's syndrome, IgG4-related disease, retroperitoneal fibrosis, juvenile idiopathic arthritis including systemic juvenile idiopathic arthritis (Still's disease), adult-onset Still's disease, ligneous conjunctivitis, Mooren's ulcer, pityriasis lichenoides et varioliformis acuta (PLEVA, also known as Mucha-Habermann disease), multifocal motor neuropathy (MMN), paediatric acute-onset neuropsychiatric syndrome (PANS) (including paediatric autoimmune neuropsychiatric disorders associated with streptococcal infections (PANDAS)), paraneoplastic syndromes (including paraneoplastic cerebellar degeneration, Lambert-Eaton myaesthenic syndrome, limbic encephalitis, brainstem encephalitis, opsoclonus myoclonus ataxia syndrome, anti-NMDA receptor encephalitis, thymoma-associated multiorgan autoimmunity), perivenous encephalomyelitis, reflex sympathetic dystrophy, relapsing polychondritis, sperm & testicular autoimmunity, Susac's syndrome, Tolosa-Hunt syndrome, Vogt-Koyanagi-Harada Disease, anti-synthetase syndrome, autoimmune enteropathy, immune dysregulation polyendocrinopathy enteropathy X-linked (IPEX), microscopic colitis, autoimmune lymphoproliferative syndrome (ALPS), autoimmune polyendocrinopathy-candidiasis-ectodermal dystrophy syndrome (APEX), gout, pseudogout, amyloid (including AA or secondary amyloidosis), eosinophilic fasciitis (Shulman syndrome) progesterone hypersensitivity (including progesterone dermatitis), familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria), Schnitzler syndrome; familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), and renal inflammatory disorders (e.g. diabetic nephropathy, membranous nephropathy, minimal change disease, crescentic glomerulonephritis, acute kidney injury, renal transplantation).

In one embodiment, the inflammatory disease or disease associated with an undesirable immune response is, or is associated with, a disease selected from the following autoinflammatory diseases: familial Mediterranean fever (FMF), tumour necrosis factor (TNF) receptor-associated periodic fever syndrome (TRAPS), hyperimmunoglobulinaemia D with periodic fever syndrome (HIDS), PAPA (pyogenic arthritis, pyoderma gangrenosum, and severe cystic acne) syndrome, deficiency of interleukin-1 receptor antagonist (DIRA), deficiency of the interleukin-36-receptor antagonist (DITRA), cryopyrin-associated periodic syndromes (CAPS) (including familial cold autoinflammatory syndrome [FCAS], Muckle-Wells syndrome, and neonatal onset multisystem inflammatory disease [NOMID]), NLRP12-associated autoinflammatory disorders (NLRP12AD), periodic fever aphthous stomatitis (PFAPA), chronic atypical neutrophilic dermatosis with lipodystrophy and elevated temperature (CANDLE), Majeed syndrome, Blau syndrome (also known as juvenile systemic granulomatosis), macrophage activation syndrome, chronic recurrent multifocal osteomyelitis (CRMO), familial cold autoinflammatory syndrome, mutant adenosine deaminase 2 and monogenic interferonopathies (including Aicardi-Goutieres syndrome, retinal vasculopathy with cerebral leukodystrophy, spondyloenchondrodysplasia, STING [stimulator of interferon genes]-associated vasculopathy with onset in infancy, proteasome associated autoinflammatory syndromes, familial chilblain lupus, dyschromatosis symmetrica hereditaria) and Schnitzler syndrome.

In one embodiment, the inflammatory disease or disease associated with an undesirable immune response is, or is associated with, a disease selected from the following diseases mediated by excess NF-κB or gain of function in the NF-κB signalling pathway or in which there is a major contribution to the abnormal pathogenesis therefrom (including non-canonical NF-κB signalling): familial cylindromatosis, congenital B cell lymphocytosis, OTULIN-related autoinflammatory syndrome, type 2 diabetes mellitus, insulin resistance and the metabolic syndrome (including obesity-associated inflammation), atherosclerotic disorders (e.g. myocardial infarction, angina, ischaemic heart failure, ischaemic nephropathy, ischaemic stroke, peripheral vascular disease, aortic aneurysm), renal inflammatory disorders (e.g. diabetic nephropathy, membranous nephropathy, minimal change disease, crescentic glomerulonephritis, acute kidney injury, renal transplantation), asthma, COPD, type 1 diabetes mellitus, rheumatoid arthritis, multiple sclerosis, inflammatory bowel disease (including ulcerative colitis and Crohn's disease), and SLE.

In one embodiment, the disease is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis, chronic obstructive pulmonary disease and asthma.

In one embodiment, the disease is multiple sclerosis.

In one embodiment, the disease is psoriasis.

In one embodiment, the disease is asthma.

In one embodiment, the disease is chronic obstructive pulmonary disease.

In one embodiment, the disease is systemic lupus erythematosus.

### Administration

The compound of formula (I) is usually administered as a pharmaceutical composition. Thus, in one embodiment, is provided a pharmaceutical composition comprising a compound of formula (I) and one or more pharmaceutically acceptable diluents or carriers.

The compound of formula (I) may be administered by any convenient method, e.g. by oral, parenteral, buccal, sublingual, nasal, rectal, intrathecal or transdermal administration, and the pharmaceutical compositions adapted accordingly.

The compound of formula (I) may be administered topically to the target organ e.g. topically to the eye, lung, nose or skin. Hence the invention provides a pharmaceutical composition comprising a compound of formula (I) optionally in combination with one or more topically acceptable diluents or carriers.

A compound of formula (I) which is active when given orally can be formulated as a liquid or solid, e.g. as a syrup, suspension, emulsion, tablet, capsule or lozenge.

A liquid formulation will generally consist of a suspension or solution of the compound of formula (I) in a suitable liquid carrier(s). Suitably the carrier is non-aqueous e.g. polyethylene glycol or an oil. The formulation may also contain a suspending agent, preservative, flavouring and/or colouring agent.

A composition in the form of a tablet can be prepared using any suitable pharmaceutical carrier(s) routinely used for preparing solid formulations, such as magnesium stearate, starch, lactose, sucrose and cellulose.

A composition in the form of a capsule can be prepared using routine encapsulation procedures, e.g. pellets containing the active ingredient can be prepared using standard carriers and then filled into a hard gelatine capsule; alternatively, a dispersion or suspension can be prepared using any suitable pharmaceutical carrier(s), e.g. aqueous gums, celluloses, silicates or oils and the dispersion or suspension then filled into a soft gelatine capsule.

Typical parenteral compositions consist of a solution or suspension of the compound of formula (I) in a sterile aqueous carrier or parenterally acceptable oil, e.g. polyethylene glycol, polyvinyl pyrrolidone, lecithin, arachis oil or sesame oil. Alternatively, the solution can be lyophilised and then reconstituted with a suitable solvent just prior to administration.

Compositions for nasal administration may conveniently be formulated as aerosols, drops, gels and powders. Aerosol formulations typically comprise a solution or fine suspension of the compound of formula (I) in a pharmaceutically acceptable aqueous or non-aqueous solvent and are usually presented in single or multidose quantities in sterile form in a sealed container which can take the form of a cartridge or refill for use with an atomising device. Alternatively, the sealed container may be a disposable dispensing device such as a single dose nasal inhaler or an aerosol dispenser fitted with a metering valve. Where the dosage form comprises an aerosol dispenser, it will contain a propellant which can be a compressed gas e.g. air, or an organic propellant such as a chlorofluorocarbon (CFC) or a hydrofluorocarbon (HFC). Aerosol dosage forms can also take the form of pump-atomisers.

Topical administration to the lung may be achieved by use of an aerosol formulation. Aerosol formulations typically comprise the active ingredient suspended or dissolved in a suitable aerosol propellant, such as a chlorofluorocarbon (CFC) or a hydrofluorocarbon (HFC).

Topical administration to the lung may also be achieved by use of a non-pressurised formulation such as an aqueous solution or suspension. These may be administered by means of a nebuliser e.g. one that can be hand-held and portable or for home or hospital use (i.e. non-portable). The formulation may comprise excipients such as water, buffers, tonicity adjusting agents, pH adjusting agents, surfactants and co-solvents.

Topical administration to the lung may also be achieved by use of a dry-powder formulation. The formulation will typically contain a topically acceptable diluent such as lactose, glucose or mannitol (preferably lactose).

The compound of the invention may also be administered rectally, for example in the form of suppositories or enemas, which include aqueous or oily solutions as well as suspensions and emulsions and foams. Such compositions are prepared following standard procedures, well known by those skilled in the art. For example, suppositories can be prepared by mixing the active ingredient with a conventional suppository base such as cocoa butter or other glycerides.

In this case, the drug is mixed with a suitable non-irritating excipient which is solid at ordinary temperatures but liquid at the rectal temperature and will therefore melt in the rectum to release the drug. Such materials are cocoa butter and polyethylene glycols.

Generally, for compositions intended to be administered topically to the eye in the form of eye drops or eye ointments, the total amount of the compound of the present invention will be about 0.0001 to less than 4.0% (w/w).

Preferably, for topical ocular administration, the compositions administered according to the present invention will be formulated as solutions, suspensions, emulsions and other dosage forms.

The compositions administered according to the present invention may also include various other ingredients, including, but not limited to, tonicity agents, buffers, surfactants, stabilizing polymer, preservatives, co-solvents and viscosity building agents. Suitable pharmaceutical compositions of the present invention include a compound of the invention formulated with a tonicity agent and a buffer. The pharmaceutical compositions of the present invention may further optionally include a surfactant and/or a palliative agent and/or a stabilizing polymer.

Various tonicity agents may be employed to adjust the tonicity of the composition, preferably to that of natural tears for ophthalmic compositions. For example, sodium chloride, potassium chloride, magnesium chloride, calcium chloride, simple sugars such as dextrose, fructose, galactose, and/or simply polyols such as the sugar alcohols mannitol, sorbitol, xylitol, lactitol, isomaltitol, maltitol, and hydrogenated starch hydrolysates may be added to the composition to approximate physiological tonicity. Such an amount of tonicity agent will vary, depending on the particular agent to be added. In general, however, the compositions will have a tonicity agent in an amount sufficient to cause the final composition to have an ophthalmically acceptable osmolality (generally about 150-450 mOsm, preferably 250-350 mOsm and most preferably at approximately 290 mOsm). In general, the tonicity agents of the invention will be present in the range of 2 to 4% w/w. Preferred tonicity agents of the invention include the simple sugars or the sugar alcohols, such as D-mannitol.

An appropriate buffer system (e.g. sodium phosphate, sodium acetate, sodium citrate, sodium borate or boric acid) may be added to the compositions to prevent pH drift under storage conditions. The particular concentration will vary, depending on the agent employed. Preferably however, the buffer will be chosen to maintain a target pH within the range of pH 5 to 8, and more preferably to a target pH of pH 5 to 7.

Surfactants may optionally be employed to deliver higher concentrations of compound of the present invention. The surfactants function to solubilise the compound and stabilise colloid dispersion, such as micellar solution, microemulsion, emulsion and suspension. Examples of surfactants which may optionally be used include polysorbate, poloxamer, polyosyl 40 stearate, polyoxyl castor oil, tyloxapol, Triton, and sorbitan monolaurate. Preferred surfactants to be employed in the invention have a hydrophile/lipophile/balance “HLB” in the range of 12.4 to 13.2 and are acceptable for ophthalmic use, such as TritonX114 and tyloxapol.

Additional agents that may be added to the ophthalmic compositions of compounds of the present invention are demulcents which function as a stabilising polymer. The stabilizing polymer should be an ionic/charged example with precedence for topical ocular use, more specifically, a polymer that carries negative charge on its surface that can exhibit a zeta-potential of (−)10-50 mV for physical stability and capable of making a dispersion in water (i.e. water soluble). A preferred stabilising polymer of the invention would be polyelectrolyte, or polyelectrolytes if more than one, from the family of cross-linked polyacrylates, such as carbomers and Pemulen®, specifically Carbomer 974p (polyacrylic acid), at 0.1-0.5% w/w.

Other compounds may also be added to the ophthalmic compositions of the compound of the present invention to increase the viscosity of the carrier. Examples of viscosity enhancing agents include, but are not limited to: polysaccharides, such as hyaluronic acid and its salts, chondroitin sulfate and its salts, dextrans, various polymers of the cellulose family; vinyl polymers; and acrylic acid polymers.

Topical ophthalmic products are typically packaged in multidose form. Preservatives are thus required to prevent microbial contamination during use. Suitable preservatives include: benzalkonium chloride, chlorobutanol, benzododecinium bromide, methyl paraben, propyl paraben, phenylethyl alcohol, edentate disodium, sorbic acid, polyquaternium-1, or other agents known to those skilled in the art. Such preservatives are typically employed at a level of from 0.001 to 1.0% w/v. Unit dose compositions of the present invention will be sterile, but typically unpreserved. Such compositions, therefore, generally will not contain preservatives.

Compositions suitable for buccal or sublingual administration include tablets, lozenges and pastilles where the compound of formula (I) is formulated with a carrier such as sugar and acacia, tragacanth, or gelatine and glycerine.

Compositions suitable for transdermal administration include ointments, gels and patches.

The composition may contain from 0.1% to 100% by weight, for example from 10% to 60% by weight, of the compound of formula (I), depending on the method of administration. The composition may contain from 0% to 99% by weight, for example 40% to 90% by weight, of the carrier, depending on the method of administration. The composition may contain from 0.05 mg to 1000 mg, for example from 1.0 mg to 500 mg, such as from 1.0 mg to 50 mg, e.g. about 10 mg of the compound of formula (I), depending on the method of administration. The composition may contain from 50 mg to 1000 mg, for example from 100 mg to 400 mg of the carrier, depending on the method of administration. The dose of the compound used in the treatment of the aforementioned disorders will vary in the usual way with the seriousness of the disorders, the weight of the sufferer, and other similar factors. However, as a general guide suitable unit doses may be 0.05 mg to 1000 mg, more suitably 1.0 mg to 500 mg, such as from 1.0 mg to 50 mg, e.g. about 10 mg and such unit doses may be administered more than once a day, for example two or three times a day. Such therapy may extend for a number of weeks or months.

In one embodiment of the invention, the compound of formula (I) is used in combination with a further therapeutic agent or agents. When the compound of formula (I) is used in combination with other therapeutic agents, the compounds may be administered either sequentially or simultaneously by any convenient route. Alternatively, the compounds may be administered separately.

Therapeutic agents which may be used in combination with the present invention include: corticosteroids (glucocorticoids), retinoids (e.g. acitretin, isotretinoin, tazarotene), anthralin, vitamin D analogues (e.g. cacitriol, calcipotriol), calcineurin inhibitors (e.g. tacrolimus, pimecrolimus), phototherapy or photochemotherapy (e.g. psoralen ultraviolet irradiation, PUVA) or other form of ultraviolet light irradiation therapy, ciclosporine, thiopurines (e.g. azathioprine, 6-mercaptopurine), methotrexate, anti-TNFα agents (e.g. infliximab, etanercept, adalimumab, certolizumab, golimumab and biosimilars), phosphodiesterase-4 (PDE4) inhibition (e.g. apremilast, crisaborole), anti-IL-17 agents (e.g. brodalumab, ixekizumab, secukinumab), anti-IL12/IL-23 agents (e.g. ustekinumab, briakinumab), anti-IL-23 agents (e.g. guselkumab, tildrakizumab), JAK (Janus Kinase) inhibitors (e.g. tofacitinib, ruxolitinib, baricitinib, filgotinib, upadacitinib), plasma exchange, intravenous immune globulin (IVIG), cyclophosphamide, anti-CD20 B cell depleting agents (e.g. rituximab, ocrelizumab, ofatumumab, obinutuzumab), anthracycline analogues (e.g. mitoxantrone), cladribine, sphingosine 1-phosphate receptor modulators or sphingosine analogues (e.g. fingolimod, siponimod, ozanimod, etrasimod), interferon beta preparations (including interferon beta 1b/1a), glatiramer, anti-CD3 therapy (e.g. OKT3), anti-CD52 targeting agents (e.g. alemtuzumab), leflunomide, teriflunomide, gold compounds, laquinimod, potassium channel blockers (e.g. dalfampridine/4-aminopyridine), mycophenolic acid, mycophenolate mofetil, purine analogues (e.g. pentostatin), mTOR (mechanistic target of rapamycin) pathway inhibitors (e.g. sirolimus, everolimus), anti-thymocyte globulin (ATG), IL-2 receptor (CD25) inhibitors (e.g. basiliximab, daclizumab), anti-IL-6 receptor or anti-IL-6 agents (e.g. tocilizumab, siltuximab), Bruton's tyrosine kinase (BTK) inhibitors (e.g. ibrutinib), tyrosine kinase inhibitors (e.g. imatinib), ursodeoxycholic acid, hydroxychloroquine, chloroquine, B cell activating factor (BAFF, also known as BLyS, B lymphocyte stimulator) inhibitors (e.g. belimumab, blisibimod), other B cell targeted therapy including fusion proteins targeting both APRIL (A PRoliferation-Inducing Ligand) and BLyS (e.g. atacicept), PI3K inhibitors including pan-inhibitors or those targeting the p110δ and/or p110γ containing isoforms (e.g. idelalisib, copanlisib, duvelisib), interferon α receptor inhibitors (e.g. anifrolumab, sifalimumab), T cell co-stimulation blockers (e.g. abatacept, belatacept), thalidomide and its derivatives (e.g. lenalidomide), dapsone, clofazimine, leukotriene antagonists (e.g. montelukast), theophylline, anti-IgE therapy (e.g. omalizumab), anti-IL-5 agents (e.g. mepolizumab, reslizumab), long-acting muscarinic agents (e.g. tiotropium, aclidinium, umeclidinium), PDE4 inhibitors (e.g. roflumilast), riluzole, free radical scavengers (e.g. edaravone), proteasome inhibitors (e.g. bortezomib), complement cascade inhibitors including those directed against C5 (e.g. eculizumab), immunoadsor, antithymocyte globulin, 5-aminosalicylates and their derivatives (e.g. sulfasalazine, balsalazide, mesalamine), anti-integrin agents including those targeting α4β1 and/or α4β7 integrins (e.g. natalizumab, vedolizumab), anti-CD11-α agents (e.g. efalizumab), non-steroidal anti-inflammatory drugs (NSAIDs) including the salicylates (e.g. aspirin), propionic acids (e.g. ibuprofen, naproxen), acetic acids (e.g. indomethacin, diclofenac, etodolac), oxicams (e.g. meloxicam) and fenamates (e.g. mefenamic acid), selective or relatively selective COX-2 inhibitors (e.g. celecoxib, etroxicoxib, valdecoxib and etodolac, meloxicam, nabumetone), colchicine, IL-4 receptor inhibitors (e.g. dupilumab), topical/contact immunotherapy (e.g. diphenylcyclopropenone, squaric acid dibutyl ester), anti-IL-1 receptor therapy (e.g. anakinra), IL-1β inhibitor (e.g. canakinumab), IL-1 neutralising therapy (e.g. rilonacept), chlorambucil, specific antibiotics with immunomodulatory properties and/or ability to modulate NRF2 (e.g. tetracyclines including minocycline, clindamycin, macrolide antibiotics), anti-androgenic therapy (e.g. cyproterone, spironolactone, finasteride), pentoxifylline, ursodeoxycholic acid, obeticholic acid, fibrate, cystic fibrosis transmembrane conductance regulator (CFTR) modulators, VEGF (vascular endothelial growth factor) inhibitors (e.g. bevacizumab, ranibizumab, pegaptanib, aflibercept), pirfenidone, and mizoribine.

Compounds of formula (I) may display one or more of the following desirable properties:
low IC₅₀ values for inhibiting release of cytokines, e.g., IL-1p and/or IL-6, from cells;
low EC₅₀ and/or high Eₘₐₓ values for activating the NRF2 pathway;
high EC₅₀ and/or low Eₘₐₓ values for activating the NRF2 pathway;
enhanced efficacy through improved hydrolytic stability and/or augmented maximum response;
reduced dose and dosing frequency through improved pharmacokinetics;
improved oral systemic bioavailability;
reduced plasma clearance following intravenous dosing;
augmented cell permeability;
enhanced aqueous solubility;
good tolerability, for example, by limiting the flushing and/or gastrointestinal side effects provoked by oral DMF (Hunt T. et al., 2015; WO2014/152494A1, incorporated herein by reference), possibly by reducing or eliminating HCA2 activity;
low toxicity at the relevant therapeutic dose;
distinct anti-inflammatory profiles resulting from varied electrophilicities, leading to differential targeting of the cysteine proteome (van der Reest J. et al., 2018) and, therefore, modified effects on gene activation).

### Abbreviations

4OI 4-octyl itaconic acid
Ac acetyl
aq. aqueous
BBFO broadband fluorine observe
BEH ethylene bridged hybrid
BTEAC benzyl(triethyl)ammonium chloride
Bu butyl
ca. circa
CSH charged surface hybrid
d doublet
DAD diode array detector
DBU 1,8-diazabicyclo(5.4.0)undec-7-ene
DCC N,N′-dicyclohexylcarbodiimide
DCM dichloromethane
DIPEA N,N-diisopropylethylamine
DMAP 4-dimethylaminopyridine
DMF dimethyl fumarate
DMSO dimethyl sulfoxide
EDCI 1-ethyl-3-(3-dimethylaminopropyl)carbodiimide
Et ethyl
ES⁺ electrospray
FBS fetal bovine serum
g gram(s)
h hour(s)
HATU 1-[bis(dimethylamino)methylene]-1H-1,2,3-triazolo[4,5-b]pyridinium 3-oxid
hexafluorophosphate
HPLC high-performance liquid chromatography
LCMS liquid chromatography-mass spectrometry
LPS lipopolysaccharide
m multiplet
M molar concentration/molar mass
Me methyl
m/z mass to charge ratio
(M)Hz (mega)hertz
min(s) minute(s)
mL millilitres
mm millimeter
mmol millimole
MS mass spectrometry
MSD mass selective detector
MTBE methyl tertiary-butyl ether
nm nanometre
NMP N-methyl-2-pyrrolidone
NMR nuclear magnetic resonance
NRF2 nuclear factor (erythroid-derived 2)-like 2
O/N overnight
PDA photodiode array
ppm parts per million
rpm revolutions per minute
RT room temperature
s singlet
sat. saturated
t triplet
T3P propanephosphonic acid anhydride
TEA triethylamine
TFA trifluoroacetic acid
THF tetrahydrofuran
μL microlitre
μM micromolar
UPLC ultra performance liquid chromatography
VWD variable wavelength detector
wt. weight
° C. degrees centigrade

### EXAMPLES

### Analytical Equipment

NMR spectra were recorded using a Bruker 400 MHz Avance Ill spectrometer fitted with a BBFO 5 mm probe, or a Bruker 500 MHz Avance Ill HD spectrometer equipped with a Bruker 5 mm SmartProbe™. Spectra were measured at 298 K, unless indicated otherwise, and were referenced relative to the solvent resonance. The chemical shifts are reported in parts per million. Data were acquired using Bruker TopSpin software.

UPLC/MS analysis was carried out on a Waters Acquity UPLC system using either a Waters Acquity CSH C18 or BEH C18 column (2.1×30 mm) maintained at a temperature of 40° C. and eluted with a linear acetonitrile gradient appropriate for the lipophilicity of the compound over 3 or 10 minutes at a constant flow rate of 0.77 mL/min. The aqueous portion of the mobile phase was either 0.1% Formic Acid (CSH C18 column) or 10 mM Ammonium Bicarbonate (BEH C18 column). LC-UV chromatograms were recorded using a Waters Acquity PDA detector between 210 and 400 nm. Mass spectra were recorded using a Waters Acquity Qda detector with electrospray ionisation switching between positive and negative ion mode. Sample concentration was adjusted to give adequate UV response.

LCMS analysis was carried out on an Agilent LCMS system using either a Waters Acquity CSH C18 or BEH C18 column (4.6×30 mm) maintained at a temperature of 40° C. and eluted with a linear acetonitrile gradient appropriate for the lipophilicity of the compound over 4 or 15 minutes at a constant flow rate of 2.5 mL/min. The aqueous portion of the mobile phase was either 0.1% Formic Acid (CSH C18 column) or 10 mM Ammonium Bicarbonate (BEH C18 column). LC-UV chromatograms were recorded using an Agilent VWD or DAD detector at 254 nm. Mass spectra were recorded using an Agilent MSD detector with electrospray ionisation switching between positive and negative ion mode. Sample concentration was adjusted to give adequate UV response.

### Commercial Materials

All starting materials disclosed herein are commercially available, unless otherwise stated. Dimethyl itaconate was purchased from Sigma-Aldrich (product number: 109533). 1-Monomethyl itaconate was purchased from Enamine. 4-Octyl itaconate was purchased from BOC biosciences (product number: B0001-007866).

Unless otherwise stated all reactions were stirred. Organic solutions were routinely dried over anhydrous sodium or magnesium sulfate.

### Intermediate 1—2-methylene-4-oxo-4-(2,2,2-trichloroethoxy)butanoic acid

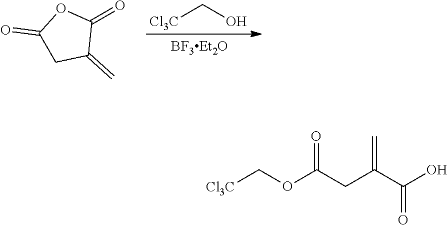

To a mixture of 3-methylenedihydrofuran-2,5-dione (5.0 g, 44.6 mmol) and 2,2,2-trichloroethanol (10.0 g, 66.1 mmol) was added boron trifluoride diethyl etherate (634 mg, 4.46 mmol), and the mixture was allowed to stir at 75° C. for 40 minutes. The reaction mixture was cooled to room temperature, quenched with methanol (2 mL), diluted with EtOAc (50 mL) and water (20 mL), separated and extracted with EtOAc (2×20 mL). The organic layers were combined and washed with brine and dried. The filtrate was concentrated under reduced pressure, the residue was purified by reversed column chromatography (Column: Boston ODS 120 g Flash; Flow Rate: 40 mL/min; solvent system: MeCN/(10 mmol/L HCl/water); MeCN gradient: 60-80%; collection wavelength: 214 nm). The fractions were concentrated under reduced pressure to remove MeCN, and the residue was lyophilized to give 2-methylene-4-oxo-4-(2,2,2-trichloroethoxy)butanoic acid (9.0 g, 77% yield, purity 80%) as white solid. The solid was triturated in n-hexane (80 mL)/MTBE (8 mL) overnight, recovered by filtration, and dried at 40° C. under reduced pressure to give 2-methylene-4-oxo-4-(2,2,2-trichloroethoxy)butanoic acid (8.0 g, 68% yield). LCMS m/z 283.2 (M+Na)+ (ES+).

### Intermediate 2—1-(4-(trifluoromethyl)phenyl)cyclobutan-1-ol

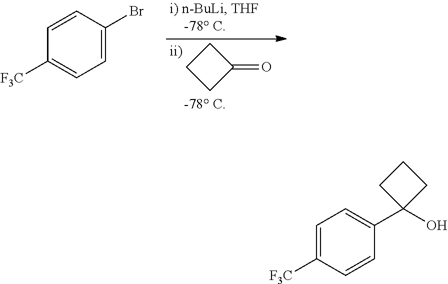 [SMILES: FC(F)(F)c1ccc(Br)cc1 ; O=C1CCC1 ; OC1(c2ccc(C(F)(F)F)cc2)CCC1]

n-BuLi solution in hexane (2.5 M, 43.7 mL, 109.2 mmol) was added to a solution of 1-bromo-4-(trifluoromethyl)benzene (22.3 g, 99.5 mmol) in THF (180 mL) at −78° C. and the mixture was stirred at −78° C. for 1 hr. Cyclobutanone (7.6 g, 109.2 mmol) was added, and the mixture was stirred at −78° C. for 5 hrs, then quenched with saturated aqueous NH₄Cl solution (200 mL). The phases were separated and the aqueous layer was extracted with MTBE (2×80 mL). The combined organic layers were washed with brine, dried and concentrated under reduced pressure. The residue was purified by flash column chromatography on silica (0-14% MTBE/petroleum ether) to give 1-(4-(trifluoromethyl)phenyl)cyclobutan-1-ol (16.5 g, 76.3 mmol, 77% yield) as a yellow oil. ¹H NMR (400 MHz, CDC₃) δ: 7.61 (s, 4H), 2.59-2.48 (m, 2H), 2.43-2.32 (m, 2H), 2.12-1.98 (m, 1H), 1.81-1.66 (m, 1H). One exchangeable proton not observed.

### Intermediate 3—1-(3-fluoro-4-(trifluoromethyl)phenyl)cyclobutan-1-ol

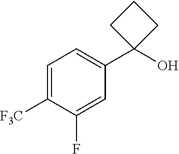 [SMILES: OC1(c2ccc(C(F)(F)F)c(F)c2)CCC1]

Prepared from 4-bromo-2-fluoro-1-(trifluoromethyl)benzene and cyclobutanone using a similar procedure to Intermediate 2. Obtained as yellow oil (3.65 g, 54% yield). ¹H NMR (400 MHz, CDCl₃) δ: 7.60 (t, J=8.0 Hz, 1H), 7.40-7.35 (m, 2H), 2.57-2.50 (m, 2H), 2.44-2.37 (m, 2H), 2.11-2.09 (m, 1H), 1.80-1.77 (m, 1H).

### Intermediate 4—3-(4-(trifluoromethyl)phenyl)oxetan-3-ol

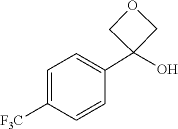 [SMILES: OC1(c2ccc(C(F)(F)F)cc2)COC1]

Prepared from 1-bromo-4-(trifluoromethyl)benzene and oxetan-3-one using a similar procedure to Intermediate 2. Obtained as yellow oil (700 mg, 58% yield). ¹H NMR (400 MHz, CDCl₃) δ: 7.78 (d, J=8.4 Hz, 2H), 7.69 (d, J=8.4 Hz, 2H), 4.95 (d, J=7.6 Hz, 2H), 4.89 (d, J=7.6 Hz, 2H).

### Intermediate 5—1-(5-(trifluoromethyl)pyridin-2-yl)cyclobutan-1-ol

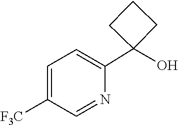 [SMILES: OC1(c2ccc(C(F)(F)F)cn2)CCC1]

Prepared from 2-bromo-5-(trifluoromethyl)pyridine and cyclobutanone using a similar procedure to Intermediate 2. Obtained as yellow oil (250 mg, 65% yield). ¹H NMR (400 MHz, CDCl₃) δ: 8.81 (s, 1H), 7.99 (dd, J=8.3, 2.3 Hz, 1H), 7.72 (d, J=8.3 Hz, 1H), 4.73 (s, 1H), 2.61-2.49 (m, 4H), 2.17-2.07 (m, 1H), 1.98-1.86 (m, 1H).

### Intermediate 6—1-(5-(trifluoromethyl)thiophen-2-yl)cyclobutan-1-ol

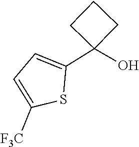 [SMILES: OC1(c2ccc(C(F)(F)F)s2)CCC1]

Prepared from 2-bromo-5-(trifluoromethyl)thiophene and cyclobutanone using a similar procedure to Intermediate 2. Obtained as yellow oil (200 mg, 67% yield)¹H NMR (400 MHz, CDCl₃) δ: 7.30-7.29 (m, 1H), 7.00-6.99 (m, 1H), 2.56-2.41 (m, 4H), 2.03-1.95 (m, 1H), 1.82-1.75 (m, 1H).

### Intermediate 7—6-(trifluoromethyl)-1,2,3,4-tetrahydronaphthalen-1-ol

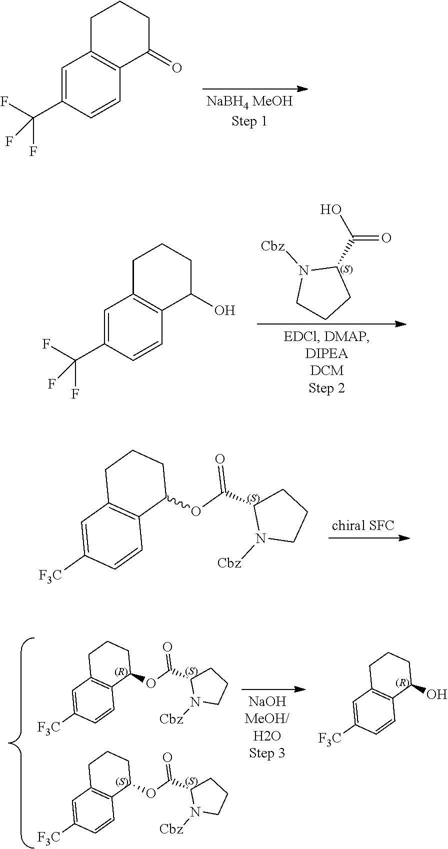

### Step 1

Sodium borohydride (710 mg, 18.68 mmol) was added at 0° C. to a solution of 6-(trifluoromethyl)-3,4-dihydronaphthalen-1(2H)-one (2.0 g, 9.3 mmol) in MeOH (50 mL), and the mixture was allowed to stir at room temperature for 0.5 hr. The reaction mixture was concentrated under reduced pressure, quenched with water (50 mL) and extracted with EtOAc (2×60 mL). The combined organic layers were washed with brine and dried. The filtrate was concentrated under reduced pressure and the residue was purified by flash column chromatography on silica (25 g, 0-20% MTBE/petroleum ether) to give 6-(trifluoromethyl)-1,2,3,4-tetrahydronaphthalen-1-ol (2.0 g, 99% yield) as white solid. LCMS m/z 199.0 (M-OH)+(ES+).

### Step 2

To the solution of 6-(trifluoromethyl)-1,2,3,4-tetrahydronaphthalen-1-ol (1.0 g, 4.6 mmol), (S)-1-(benzyloxycarbonyl)pyrrolidine-2-carboxylic acid (1.7 g, 6.9 mmol), and DMAP (847 mg, 6.9 mmol) in DCM (30 mL) was added EDCI (1.78 g, 9.3 mmol) and DIPEA (2.4 g, 18.5 mmol) at 0° C., and the resulting mixture was stirred at room temperature overnight. The reaction mixture was quenched with aq HCl (0.5 N) and the pH adjusted to 5. The organic layer was separated and the aqueous layer extracted with DCM (2×40 mL). The combined organic layers were washed with brine, dried and the filtrate was concentrated under reduced pressure. The residue was purified by flash column chromatography (0-10% MTBE/petroleum ether) to give (S)-1-benzyl 2-(6-(trifluoromethyl)-1,2,3,4-tetrahydronaphthalen-1-yl) pyrrolidine-1,2-dicarboxylate (1.9 g, 91% yield) as yellow oil. LCMS m/z 470.0 (M+Na)+ (ES+).

(S)-1-benzyl 2-(6-(trifluoromethyl)-1,2,3,4-tetrahydronaphthalen-1-yl) pyrrolidine-1,2-dicarboxylate (1.90 g, 3.00 mmol) was separated by SFC (Column: CHIRALPAK AD-5 (30*250 mm 5 μm) (Daicel); Column temperature: 35° C. CO₂ flow Rate: 36 mL/min; co solvent flow rate: 9 mL/min; total flow rate: 45 mL/min. Co solvent: MeOH. Collection wavelength: 215 nm)). The SFC fractions were concentrated under reduced pressure to give (S)-1-benzyl 2-(6-(trifluoromethyl)-1,2,3,4-tetrahydronaphthalen-1-yl) pyrrolidine-1,2-dicarboxylate ISOMER 1 (890 mg, 99 ee %, 46% yield) and (S)-1-benzyl 2-(6-(trifluoromethyl)-1,2,3,4-tetrahydronaphthalen-1-yl) pyrrolidine-1,2-dicarboxylate ISOMER 2 (800 mg, 100 ee %, 42% yield). The absolute configuration was arbitrarily assigned in the synthetic scheme.

Chiral HPLC: (Column: CHIRALPAKAD-3(4.6*100 mm)); Flow Rate: 2 mL/min; Co_solvent: 15% MeOH; collection wavelength: 200-400 nm) ISOMER 1: Rt=1.764 min; ISOMER 2: Rt=2.137 min.

### Step 3

A solution of (S)-1-benzyl 2-(6-(trifluoromethyl)-1,2,3,4-tetrahydronaphthalen-1-yl) pyrrolidine-1,2-dicarboxylate (ISOMER 1, 890 mg, 2.0 mmol) and NaOH (159 mg, 4.0 mmol) in MeOH/H₂O (5 mL/2.5 mL) was stirred at room temperature overnight. The mixture was concentrated under reduced pressure and pH was adjusted to 5 with addition of aq HCl (0.5 N). The organic layer was separated and the aqueous layer extracted with EtOAc (2×5 mL). The combined organic layers were washed with brine, dried over Na₂SO₄ and filtered. The filtrate was concentrated under reduced pressure to give 6-(trifluoromethyl)-1,2,3,4-tetrahydronaphthalen-1-ol ISOMER 1 (400 mg, 93% yield) as yellow solid. LCMS m/z 199.2 (M−OH)+ (ES+).

### Example 1—(S)-3-((1-(4-(trifluoromethyl)phenyl)ethoxy)carbonyl)but-3-enoic acid

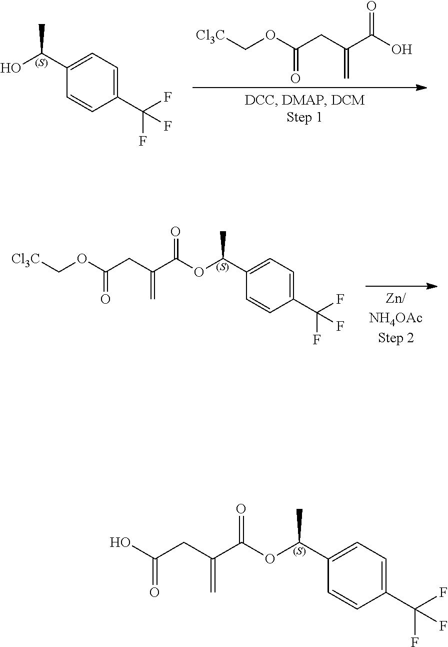

### Step 1

A mixture of (S)-1-(4-(trifluoromethyl)phenyl)ethanol (190 mg, 1.0 mmol), 2-methylene-4-oxo-4-(2,2,2-trichloroethoxy)butanoic acid (260 mg, 1.0 mmol), DCC (309 mg, 1.5 mmol) and DMAP (24 mg, 0.2 mmol) in DCM (7 mL) was stirred at room temperature for 1 hr. The mixture was filtered, the filtrate concentrated under reduced pressure, and the residue purified by flash column chromatography on silica (0-10% MTBE/petroleum ether) to give (S)-4-(2,2,2-trichloroethyl) 1-(1-(4-(trifluoromethyl)phenyl)ethyl) 2-methylenesuccinate (270 mg, 62% yield) as a light yellow oil. LCMS m/z 454.8 (M+Na)+ (ES+).

### Step 2

A mixture of (S)-4-(2,2,2-trichloroethyl) 1-(1-(4-(trifluoromethyl)phenyl)ethyl) 2-methylenesuccinate (270 mg, 0.6 mmol), zinc powder (201 mg, 3.1 mmol) and NH₄OAc (477 mg, 6.2 mmol) in THF (2.5 mL) and H₂O (0.8 mL) was stirred at room temperature for 4 hrs. The reaction mixture was filtered and the filtrate was acidified to pH 4-5 with aq HCl (0.5 N) and extracted with tert-butyl methyl ether (2×5 mL). The combined organic phases were washed with brine, dried (magnesium sulfate) and concentrated under reduced pressure. The residue was purified by prep-HPLC (Column: Waters SUNFIRE Prep C18 OBD 10 μm 19×250 mm; Flow Rate: 20 mL/min; solvent system: MeCN/(0.2% trifluoroacetic acid/water) MeCN gradient: 50-95%; collection wavelength: 214 nm). The fractions were concentrated under reduced pressure to remove MeCN, and the residue was lyophilized to give (S)-3-((1-(4-(trifluoromethyl)phenyl)ethoxy)carbonyl)but-3-enoic acid (58.1 mg, 30% yield) as yellow oil. LCMS m/z 324.9 (M+Na)+ (ES+).

¹H NMR (400 MHz, DMSO-d₆) δ: 12.40 (br, 1H), 7.73 (d, J=8.4 Hz, 2H), 7.61 (d, J=8.4 Hz, 2H), 6.26 (d, J=0.8 Hz, 1H), 5.93 (q, J=6.8 Hz, 1H), 5.83 (s, 1H), 3.31 (s, 2H), 1.50 (d, J=6.4 Hz, 3H).

### Example 2—3-((1-(4-(trifluoromethyl)phenyl)cyclobutoxy)carbonyl)but-3-enoic acid

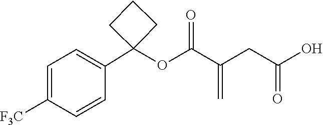 [SMILES: C=C(CC(=O)O)C(=O)OC1(c2ccc(C(F)(F)F)cc2)CCC1]

Prepared from Intermediate 1 and Intermediate 2 using a similar procedure to (S)-3-((1-(4-(trifluoromethyl)phenyl)ethoxy)carbonyl)but-3-enoic acid.

### Step 1

4-(2,2,2-trichloroethyl) 1-(1-(4-(trifluoromethyl)phenyl)cyclobutyl) 2-methylenesuccinate (260 mg, 61% yield) was obtained as a colorless oil. LCMS m/z 480.8 (M+Na)+ (ES+).

### Step 2

3-((1-(4-(trifluoromethyl)phenyl)cyclobutoxy)carbonyl)but-3-enoic acid (82.5 mg, 45% yield) was obtained as white solid. LCMS m/z 351.0 (M+Na)+ (ES+). ¹H NMR (400 MHz, DMSO-d6) δ: 12.39 (s, 1H), 7.69 (d, J=8.4 Hz, 2H), 7.63 (d, J=8.4 Hz, 2H), 6.15 (s, 1H), 5.75 (s, 1H), 3.25 (s, 2H), 2.61-2.51 (m, 4H), 1.99-1.92 (m, 1H), 1.79-1.72 (m, 1H).

### Example 3—3-((1-(3-fluoro-4-(trifluoromethyl)phenyl)cyclobutoxy)carbonyl)but-3-enoic acid

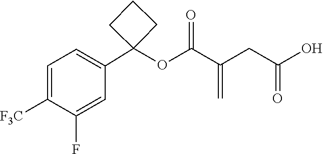 [SMILES: C=C(CC(=O)O)C(=O)OC1(c2ccc(C(F)(F)F)c(F)c2)CCC1]

Prepared from Intermediate 1 and Intermediate 3 using a similar procedure to (S)-3-((1-(4-(trifluoromethyl)phenyl)ethoxy)carbonyl)but-3-enoic acid.

### Step 1

1-(1-(3-fluoro-4-(trifluoromethyl)phenyl)cyclobutyl) 4-(2,2,2-trichloroethyl) 2-methylenesuccinate (490 mg, 52% yield) was obtained as a colorless oil. LCMS m/z 498.8 (M+Na)+ (ES+).

### Step 2

3-((1-(3-fluoro-4-(trifluoromethyl)phenyl)cyclobutoxy)carbonyl)but-3-enoic acid (134.2 mg, 38% yield) was obtained as white solid. LCMS m/z 368.8 (M+Na)+ (ES+).

¹H NMR (400 MHz, DMSO-d6) δ: 12.42 (s, 1H), 7.76 (t, J=8.0 Hz, 1H), 7.53 (d, J=12.0 Hz, 1H), 7.48 (d, J=8.0 Hz, 1H), 6.20 (s, 1H), 5.79 (s, 1H), 3.30 (s, 2H), 2.64-2.53 (m, 4H), 2.00-1.94 (m, 1H), 1.85-1.78 (m, 1H).

### Example 4—3-(((3-(4-(trifluoromethyl)phenyl)oxetan-3-yl)oxy)carbonyl)but-3-enoic acid

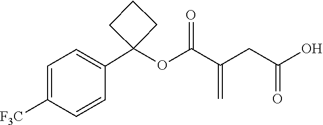 [SMILES: C=C(CC(=O)O)C(=O)OC1(c2ccc(C(F)(F)F)cc2)CCC1]

Prepared from Intermediate 1 and Intermediate 4 using a similar procedure to (S)-3-((1-(4-(trifluoromethyl)phenyl)ethoxy)carbonyl)but-3-enoic acid.

### Step 1

4-(2,2,2-trichloroethyl) 1-(3-(4-(trifluoromethyl)phenyl)oxetan-3-yl) 2-methylenesuccinate (440 mg, 52% yield) was obtained as a colorless oil. LCMS m/z 460.8 (M+H)+(ES+).

### Step 2

3-(((3-(4-(trifluoromethyl)phenyl)oxetan-3-yl)oxy)carbonyl)but-3-enoic acid (86.2 mg, 30% yield) was obtained as white solid. LCMS m/z 331.0 (M+H)+(ES+). ¹H NMR (400 MHz, DMSO-d6) δ: 12.50 (br, 1H), 7.79 (d, J=8.4 Hz, 2H), 7.74 (d, J=8.4 Hz, 2H), 6.34 (s, 1H), 5.92 (s, 1H), 4.96 (d, J=8.0 Hz, 2H), 4.87 (d, J=8.0 Hz, 2H), 3.36 (s, 2H).

### Example 5—3-((1-(5-(trifluoromethyl)pyridin-2-yl)cyclobutoxy)carbonyl)but-3-enoic acid

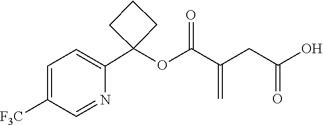 [SMILES: C=C(CC(=O)O)C(=O)OC1(c2ccc(C(F)(F)F)cn2)CCC1]

Prepared from Intermediate 1 and Intermediate 5 using a similar procedure to (S)-3-((1-(4-(trifluoromethyl)phenyl)ethoxy)carbonyl)but-3-enoic acid.

### Step 1

4-(2,2,2-trichloroethyl) 1-(1-(5-(trifluoromethyl)pyridin-2-yl)cyclobutyl) 2-methylenesuccinate (360 mg, 42% yield) was obtained as a colorless oil. LCMS m/z 460.0 (M+H)+ (ES+).

### Step 2

3-((1-(5-(trifluoromethyl)pyridin-2-yl)cyclobutoxy)carbonyl)but-3-enoic acid (72.2 mg, 28% yield) was obtained as white solid. LCMS m/z 330.0 (M+H)+ (ES+). ¹H NMR (400 MHz, DMSO-d6) δ: 12.43 (s, 1H), 8.99 (t, J=1.2 Hz, 1H), 8.16 (dd, J=8.4, 2.0 Hz, 1H), 7.55 (d, J=8.4 Hz, 1H), 6.25 d, J=0.8 Hz, 1H), 5.84 (d, J=0.8 Hz, 1H), 3.33 (s, 2H), 2.75-2.69 (m, 2H), 2.55-2.47 (m, 2H), 2.01-1.92 (m, 2H).

### Example 6—3-((1-(5-(trifluoromethyl)thiophen-2-yl)cyclobutoxy)carbonyl)but-3-enoic acid

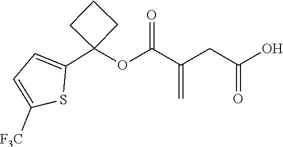 [SMILES: C=C(CC(=O)O)C(=O)OC1(c2ccc(C(F)(F)F)s2)CCC1]

Prepared from Intermediate 1 and Intermediate 6 using a similar procedure to (S)-3-((1-(4-(trifluoromethyl)phenyl)ethoxy)carbonyl)but-3-enoic acid.

### Step 1

1-(1-(5-(trifluoromethyl)thiophen-2-yl)cyclobutyl) 2-methylenesuccinate (280 mg, 67% yield) was obtained as a colorless oil. LCMS m/z 486.7 (M+H)+ (ES+).

### Step 2

3-((1-(5-(trifluoromethyl)thiophen-2-yl)cyclobutoxy)carbonyl)but-3-enoic acid (86.0 mg, 43% yield) was obtained as white solid. LCMS m/z 357.1 (M+Na)+ (ES+). ¹H NMR (400 MHz, DMSO-d6) δ: 12.39 (s, 1H), 7.60 (dd, J=4.0, 1.2 Hz, 1H), 7.32 (d, J=4.0 Hz, 1H), 6.17 (d, J=1.2 Hz, 1H), 5.80 (d, J=0.8 Hz, 1H), 3.27 (s, 2H), 2.67-2.57 (m, 4H), 1.97-1.97 (m, 1H), 1.84-1.77 (m, 3H).

### Example 7—3-(((6-(trifluoromethyl)-1,2,3,4-tetrahydronaphthalen-1-yl)oxy)carbonyl)but-3-enoic acid

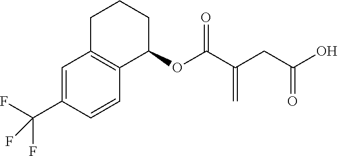 [SMILES: C=C(CC(=O)O)C(=O)O[C@@H]1CCCc2cc(C(F)(F)F)ccc21]

Prepared from Intermediate 1 and Intermediate 7 using a similar procedure to (S)-3-((1-(4-(trifluoromethyl)phenyl)ethoxy)carbonyl)but-3-enoic acid.

### Step 1

4-(2,2,2-trichloroethyl) 1-(6-(trifluoromethyl)-1,2,3,4-tetrahydronaphthalen-1-yl) 2-methylenesuccinate (300 mg, 71% yield) was obtained as a colorless oil. LCMS m/z 481.0 (M+Na)+ (ES+).

### Step 2

3-(((6-(trifluoromethyl)-1,2,3,4-tetrahydronaphthalen-1-yl)oxy)carbonyl)but-3-enoic acid (102.4 mg, 48% yield) was obtained as white solid. The absolute configuration was arbitrarily assigned. LCMS m/z 351.1 (M+Na)+ (ES+). ¹H NMR (400 MHz, DMSO-d6) δ: 12.39 (br, 1H), 7.54-7.50 (m, 2H), 7.43 (d, J=8.0 Hz, 1H), 6.16 (d, J=1.2 Hz, 1H), 5.99 (t, J=4.4 Hz, 1H), 5.78 (d, J=0.8 Hz, 1H), 3.28 (s, 2H), 2.95-2.88 (m, 1H), 2.83-2.75 (m, 1H), 2.05-1.98 (m, 1H), 1.92-1.78 (m, 1H).

### Example 8—4-methyl 1-(1-(4-(trifluoromethyl)phenyl)cyclobutyl) 2-methylenesuccinate

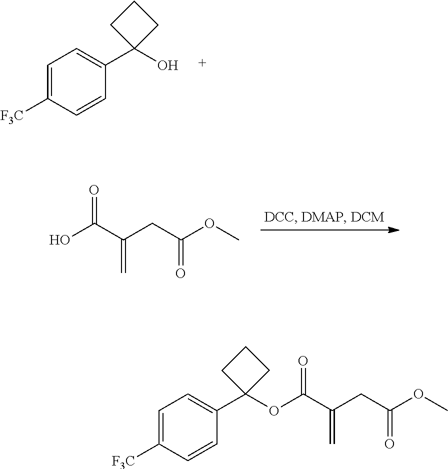

DCC (408 mg, 2.0 mmol) was added to a mixture of 1-(4-(trifluoromethyl)phenyl)cyclobutan-1-ol (Intermediate 2, 0.3 g, 1.3 mmol), 4-methoxy-2-methylene-4-oxobutanoic acid (200 mg, 1.3 mmol) and DMAP (16 mg, 0.1 mmol) in DCM (4 mL) at 0° C. The mixture was stirred at RT for 2 hrs, filtered, and the filtrate concentrated under reduced pressure. The crude product was purified by chromatography on silica (0-25% MTBE/heptane) to afford 4-methyl 1-(1-(4-(trifluoromethyl)phenyl)cyclobutyl) 2-methylenesuccinate (241 mg, 0.7 mmol) as a colourless oil. ¹H NMR (400 MHz, DMSO-d6) δ 7.74 (d, J=8.3 Hz, 2H), 7.64 (d, J=8.2 Hz, 2H), 6.25-6.20 (m, 1H), 5.86-5.81 (m, 1H), 3.59 (s, 3H), 3.38 (s, 2H), 2.66-2.52 (m, 4H), 2.05-1.91 (m, 1H), 1.85-1.71 (m, 1H).

### Example 9—1-(4-(trifluoromethyl)phenyl)cyclobutyl 4-(methylamino)-2-methylene-4-oxobutanoate

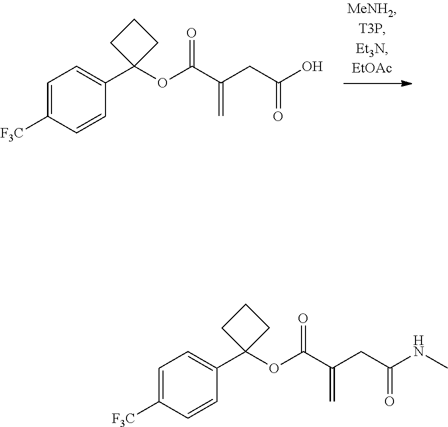 [SMILES: C=C(CC(=O)O)C(=O)OC1(c2ccc(C(F)(F)F)cc2)CCC1 ; C=C(CC(=O)NC)C(=O)OC1(c2ccc(C(F)(F)F)cc2)CCC1]

A solution of T3P (50 wt % in EtOAc, 0.8 mL, 1.4 mmol) was added dropwise to a stirred solution of 3-((1-(4-(trifluoromethyl)phenyl)cyclobutoxy)carbonyl)but-3-enoic acid (Example 2, 250 mg, 0.7 mmol), methylamine (2 M in THF, 0.5 mL, 1.0 mmol) and triethylamine (0.2 mL, 1.4 mmol) in EtOAc (3 mL) at RT. The mixture was stirred at RT for 1 hr then diluted with brine (20 mL) and extracted with EtOAc (2×20 mL). The combined organic phases were dried (magnesium sulfate) and concentrated under reduced pressure. The residue was purified by chromatography on Reverse Phase Flash C18 (5-75% MeCN/(0.1% Formic acid in Water)) to afford 1-(4-(trifluoromethyl)phenyl)cyclobutyl 4-(methylamino)-2-methylene-4-oxobutanoate (183 mg, 0.5 mmol) as a white solid. LCMS m/z 364.3 (M+Na)⁺ (ES⁺). ¹H NMR (400 MHz, DMSO-d6) δ 7.84-7.77 (m, 1H), 7.72 (d, J=8.3 Hz, 2H), 7.64 (d, J=8.3 Hz, 2H), 6.20-6.10 (m, 1H), 5.75-5.66 (m, 1H), 3.11 (s, 2H), 2.63-2.53 (m, 7H), 2.06-1.90 (m, 1H), 1.86-1.69 (m, 1H).

### Example 10—1-(4-(trifluoromethyl)phenyl)cyclobutyl 4-(dimethylamino)-2-methylene-4-oxobutanoate

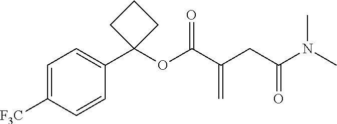 [SMILES: C=C(CC(=O)N(C)C)C(=O)OC1(c2ccc(C(F)(F)F)cc2)CCC1]

Prepared by an analogous method to Example 10 using dimethylamine (2 M in THF). Obtained as a colourless oil (202 mg, 54%). LCMS m/z 378.3 (M+Na)⁺ (ES⁺). ¹H NMR (400 MHz, DMSO) δ 7.72 (d, J=8.4 Hz, 2H), 7.65 (d, J=8.3 Hz, 2H), 6.15-6.09 (m, 1H), 5.65-5.59 (m, 1H), 3.36 (s, 2H), 2.98 (s, 3H), 2.82 (s, 3H), 2.62-2.52 (m, 4H), 2.04-1.90 (m, 1H), 1.86-1.71 (m, 1H).

### Example 11—1-(4-(trifluoromethyl)phenyl)cyclobutyl 4-amino-2-methylene-4-oxobutanoate

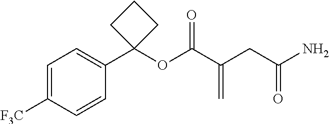 [SMILES: C=C(CC(N)=O)C(=O)OC1(c2ccc(C(F)(F)F)cc2)CCC1]

Prepared by an analogous method to Example 10 starting from ammonium chloride Obtained as a pale yellow solid (21 mg, 59% yield) LCMS m/z 350.3 (M+Na)⁺ (ES⁺). ¹H NMR (400 MHz, DMSO) δ 7.71 (d, J=8.4 Hz, 2H), 7.66 (d, J=8.4 Hz, 2H), 7.35 (s, 1H), 6.88 (s, 1H), 6.17-6.10 (m, 1H), 5.72-5.66 (m, 1H), 3.12 (s, 2H), 2.58 (t, J=7.9 Hz, 4H), 2.06-1.89 (m, 1H), 1.87-1.69 (m, 1H).

### Example 12—1-(4-(trifluoromethyl)phenyl)cyclobutyl 4-((2-(dimethylamino)ethyl)amino)-2-methylene-4-oxobutanoate

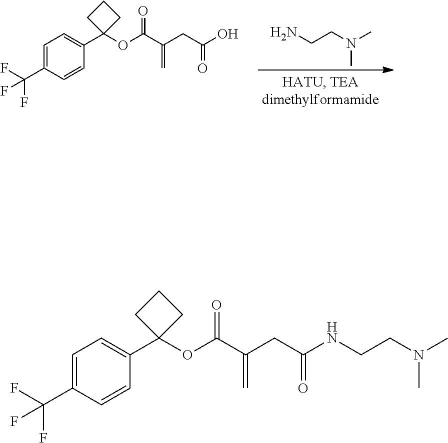

A mixture of 3-((1-(4-(trifluoromethyl)phenyl)cyclobutoxy)carbonyl)but-3-enoic acid (200 mg, 0.61 mmol), N¹,N¹-dimethylethane-1,2-diamine (54 mg, 0.61 mmol), HATU (348 mg, 0.915 mmol) and triethylamine (132 mg, 132 mmol) in DMF (3 mL) was stirred at 0° C. for 2 hrs. The reaction mixture was quenched with water (4 mL), and extracted with ethyl acetate (3×5 mL). The organic layer was washed by brine, dried over Na₂SO₄, and concentrated under reduced pressure. The residue was purified by prep-HPLC (Column: Waters X-bridge Prep C18 OBD 10 μm 19×250 mm; Flow Rate: 20 mL/min; solvent system: MeCN/(10 mmol/L NH₄HCO₃/water) gradient: MeCN: 40-95%; collection wavelength: 214 nm). The fractions were concentrated under reduced pressure to remove MeCN, and lyophilized to give 1-(4-(trifluoromethyl)phenyl)cyclobutyl 4-(2-(dimethylamino)ethylamino)-2-methylene-4-oxobutanoate (78 mg, 32% yield) as white solid. LCMS m/z 399.3 (M+H)⁺ (ES⁺). ¹H NMR (400 MHz, CDCl3) δ: 7.63-7.54 (m, 4H), 6.41 (br s, 1H), 6.37 (d, J=0.8 Hz, 1H), 5.82 (d, J=1.2 Hz, 1H), 3.30 (q, J=5.6 Hz, 2H), 3.18 (s, 2H), 2.68 (dd, J=8.6, 6.9 Hz, 4H), 2.38 (t, J=6 Hz, 2H), 2.21 (s, 6H), 2.07-1.99 (m, 1H), 1.86-1.77 (m, 1H).

### Example 13—1-(4-(trifluoromethyl)phenyl)cyclobutyl 2-methylene-4-((2-morpholinoethyl)amino)-4-oxobutanoate

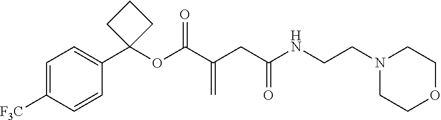 [SMILES: C=C(CC(=O)NCCN1CCOCC1)C(=O)OC1(c2ccc(C(F)(F)F)cc2)CCC1]

Prepared by an analogous method to Example 12 starting from 2-morpholinoethanamine. Obtained as a white solid (143 mg, 53% yield). LCMS m/z 441.3 (M+H)⁺ (ES⁺). ¹H NMR (400 MHz, DMSO-d6) δ: 7.85 (t, J=5.6 Hz, 1H), 7.72 (d, J=8.4 Hz, 2H), 7.65 (d, J=8.4 Hz, 2H), 6.16 (d, J=1.2 Hz, 1H), 5.73 (d, J=0.8 Hz, 1H), 3.54 (t, J=4.8 Hz, 4H), 3.17 (q, J=6 Hz, 2H), 3.13 (s, 2H), 2.62-2.55 (m, 4H), 2.33-2.29 (m, 6H), 2.00-1.94 (m, 1H), 1.83-1.75 (m, 1H).

### Example 14—1-(4-(trifluoromethyl)phenyl)cyclobutyl 2-methylene-4-((1-methylpiperidin-4-yl)amino)-4-oxobutanoate

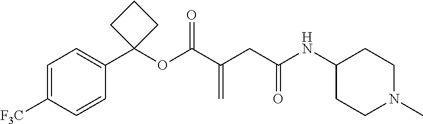 [SMILES: C=C(CC(=O)NC1CCN(C)CC1)C(=O)OC1(c2ccc(C(F)(F)F)cc2)CCC1]

Prepared by an analogous method to Example 12 starting from 1-methylpiperidin-4-amine. Obtained as a white solid (94 mg, 36% yield). LCMS m/z 425.4 (M+H)⁺ (ES⁺). ¹H NMR (400 MHz, DMSO-d6) δ: 7.83 (d, J=7.6 Hz, 1H), 7.72 (d, J=8 Hz, 2H), 7.64 (d, J=8.4 Hz, 2H), 6.13 (d, J=1.2 Hz, 1H), 5.68 (d, J=1.2 Hz, 1H), 3.52-3.43 (m, 1H), 3.12 (s, 2H), 2.69-2.60 (m, 2H), 2.60-2.57 (m, 4H), 2.12 (s, 3H), 2.00-1.87 (m, 3H), 1.82-1.75 (m, 1H), 1.69-1.60 (m, 2H), 1.42-1.32 (m, 2H).

### Example 15—1-(4-(trifluoromethyl)phenyl)cyclobutyl 4-(((1H-imidazol-2-yl)methyl)amino)-2-methylene-4-oxobutanoate

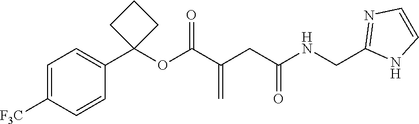 [SMILES: C=C(CC(=O)NCc1ncc[nH]1)C(=O)OC1(c2ccc(C(F)(F)F)cc2)CCC1]

Prepared by an analogous method to Example 12 starting from 1H-imidazol-2-yl)methanamine. Obtained as a white solid (40 mg, 16% yield). LCMS m/z 408.2 (M+H)⁺ (ES⁺). ¹H NMR (400 MHz, DMSO-d6) δ: 11.74 (br s, 1H), 8.40 (t, J=5.2 Hz, 1H), 7.71 (d, J=8.4 Hz, 2H), 7.63 (d, J=8.4 Hz, 2H), 6.99 (s, 1H), 6.80 (s, 1H), 6.16 (d, J=1.2 Hz, 1H), 5.73 (d, J=1.2 Hz, 1H), 4.27 (d, J=5.6 Hz, 2H), 3.20 (s, 2H), 2.56-2.51 (m, 4H), 1.97-1.92 (m, 1H), 1.79-1.74 (m, 1H).

### Example 16—1-(4-(trifluoromethyl)phenyl)cyclobutyl 4-(((1-methyl-1H-imidazol-2-yl)methyl)amino)-2-methylene-4-oxobutanoate

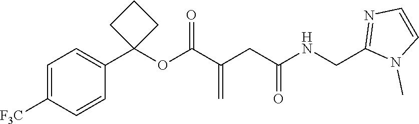 [SMILES: C=C(CC(=O)NCc1nccn1C)C(=O)OC1(c2ccc(C(F)(F)F)cc2)CCC1]

Prepared by an analogous method to Example 12 starting from 1-methyl-1H-imidazol-2-yl)methanamine. Obtained as a white solid (174 mg, 68% yield). LCMS m/z 422.2 (M+H)⁺ (ES⁺). ¹H NMR (400 MHz, DMSO-d6) δ: 8.40 (t, J=5.2 Hz, 1H), 7.72 (d, J=8.4 Hz, 2H), 7.63 (d, J=8.4 Hz, 2H), 7.05 (d, J=1.2 Hz, 1H), 6.77 (d, J=1.2 Hz s, 1H), 6.16 (d, J=1.2 Hz, 1H), 5.73 (d, J=0.8 Hz, 1H), 4.32 (d, J=5.2 Hz, 2H), 3.53 (s, 3H), 3.18 (s, 2H), 2.60-2.54 (m, 4H), 2.01-1.91 (m, 1H), 1.82-1.71 (m, 1H).

### Example 17—1-(4-(trifluoromethyl)phenyl)cyclobutyl 4-((1-amino-3-hydroxy-1-oxopropan-2-yl)amino)-2-methylene-4-oxobutanoate

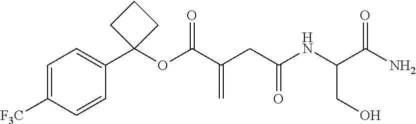 [SMILES: C=C(CC(=O)NC(CO)C(N)=O)C(=O)OC1(c2ccc(C(F)(F)F)cc2)CCC1]

Prepared by an analogous method to Example 12 starting from 2-amino-3-hydroxypropanamide. Obtained as a white solid (127 mg, 40% yield). LCMS m/z 437.6 (M+Na)⁺ (ES⁺). ¹H NMR (400 MHz, DMSO-d6) δ: 7.91 (d, J=8.1 Hz, 1H), 7.72 (d, J=8.3 Hz, 2H), 7.64 (d, J=8.2 Hz, 2H), 7.26 (s, 1H), 7.09 (s, 1H), 6.17-6.12 (m, 1H), 5.74-5.70 (m, 1H), 4.86 (t, J=5.5 Hz, 1H), 4.28-4.19 (m, 1H), 3.63-3.48 (m, 2H), 3.30-3.18 (m, 2H), 2.58 (t, J=8.7 Hz, 4H), 2.06-1.89 (m, 1H), 1.84-1.70 (m, 1H).

### Example 18—1-(4-(trifluoromethyl)phenyl)cyclobutyl 4-((1,1-dioxidothietan-3-yl)amino)-2-methylene-4-oxobutanoate

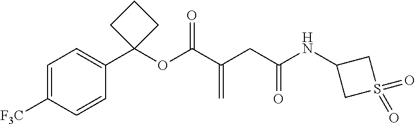 [SMILES: C=C(CC(=O)NC1CS(=O)(=O)C1)C(=O)OC1(c2ccc(C(F)(F)F)cc2)CCC1]

Prepared by an analogous method to Example 9 starting from 3-aminoheptane 1,1-dioxide. Obtained as a white solid (193 mg, 65% yield). LCMS m/z 454.2 (M+Na)⁺ (ES⁺). ¹H NMR (400 MHz, DMSO-d6) δ: 8.75 (d, J=5.3 Hz, 1H), 7.73 (d, J=8.3 Hz, 2H), 7.64 (d, J=8.2 Hz, 2H), 6.19 (d, J=1.4 Hz, 1H), 5.75 (d, J=1.4 Hz, 1H), 4.57-4.48 (m, 2H), 4.38-4.30 (m, 1H), 4.03-3.95 (m, 2H), 3.19 (s, 2H), 2.64-2.52 (m, 4H), 2.02-1.92 (m, 1H), 1.85-1.73 (m, 1H).

### Example 19—1-(4-(trifluoromethyl)phenyl)cyclobutyl 4-((2-hydroxyethyl)amino)-2-methylene-4-oxobutanoate

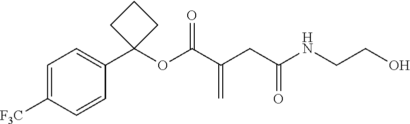 [SMILES: C=C(CC(=O)NCCO)C(=O)OC1(c2ccc(C(F)(F)F)cc2)CCC1]

Prepared by an analogous method to Example 9 starting from 2-aminoethane-1-ol. Obtained as a white solid (145 mg, 54% yield). LCMS m/z 394.2 (M+Na)⁺ (ES⁺). ¹H NMR (400 MHz, DMSO-d6) δ: 7.91 (t, J=5.7 Hz, 1H), 7.72 (d, J=8.3 Hz, 2H), 7.65 (d, J=8.2 Hz, 2H), 6.17-6.10 (m, 1H), 5.73-5.66 (m, 1H), 4.65 (t, J=5.4 Hz, 1H), 3.39 (q, J=6.0 Hz, 2H), 3.18-3.09 (m, 4H), 2.62-2.53 (m, 4H), 2.04-1.90 (m, 1H), 1.85-1.71 (m, 1H).

### Example 20—1-(4-(trifluoromethyl)phenyl)cyclobutyl 2-methylene-4-oxo-4-((tetrahydro-2H-pyran-4-yl)amino)butanoate

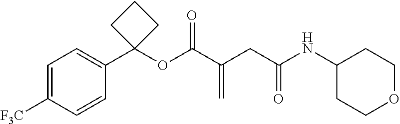 [SMILES: C=C(CC(=O)NC1CCOCC1)C(=O)OC1(c2ccc(C(F)(F)F)cc2)CCC1]

Prepared by an analogous method to Example 9 starting from tetrahydro-2H-pyran-4-amine. Obtained as a white solid (219 mg, 74% yield). LCMS m/z 434.0 (M+Na)⁺ (ES⁺). ¹H NMR (400 MHz, DMSO-d6) δ: 7.91 (d, J=7.6 Hz, 1H), 7.72 (d, J=8.3 Hz, 2H), 7.64 (d, J=8.3 Hz, 2H), 6.19-6.08 (m, 1H), 5.74-5.62 (m, 1H), 3.86-3.67 (m, 3H), 3.31-3.29 (m, 2H), 3.13 (s, 2H), 2.64-2.53 (m, 4H), 2.06-1.90 (m, 1H), 1.87-1.72 (m, 1H), 1.70-1.59 (m, 2H), 1.45-1.28 (m, 2H).

### Example 21—1-(4-(trifluoromethyl)phenyl)cyclobutyl 4-((2-amino-2-oxoethyl)amino)-2-methylene-4-oxobutanoate

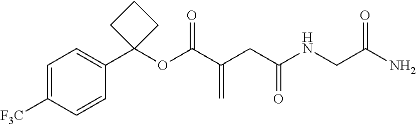 [SMILES: C=C(CC(=O)NCC(N)=O)C(=O)OC1(c2ccc(C(F)(F)F)cc2)CCC1]

Prepared by an analogous method to Example 9 starting from 2-aminoacetamide. Obtained as a white solid (35 mg, 13% yield). LCMS m/z 385.0 (M+H)⁺ (ES⁺). ¹H NMR (400 MHz, DMSO-d6) δ: 8.10 (t, J=5.8 Hz, 1H), 7.72 (d, J=8.3 Hz, 2H), 7.65 (d, J=8.3 Hz, 2H), 7.24 (s, 1H), 7.04 (s, 1H), 6.20-6.14 (m, 1H), 5.78-5.69 (m, 1H), 3.64 (d, J=5.7 Hz, 2H), 3.21 (s, 2H), 2.58 (t, J=7.9 Hz, 4H), 2.04-1.89 (m, 1H), 1.83-1.69 (m, 1H).

### Example 22—2-((3-((1-(4-(trifluoromethyl)phenyl)cyclobutoxy)carbonyl)but-3-enoyl)oxy)acetic acid

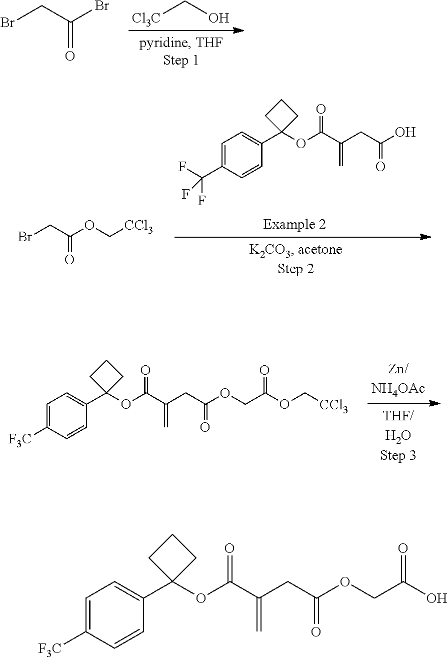

### Step 1

To a solution of ethyl 2,2,2-trichloroethan-1-ol (8.9 g, 60 mmol) and pyridine (6 g, 75 mmol) in THF (150 mL) was added 2-bromoacetyl bromide (10 g, 50 mmol) at 0° C., and the reaction mixture was stirred at room temperature for 4 hrs. The reaction mixture was quenched with water (400 mL) and extracted with MTBE (3×100 mL). The combined organic layers were washed with aq HCl aq. (0.5N, 2×50 mL) and brine, dried (magnesium sulfate) and concentrated under reduced pressure. The residue was purified by column chromatography on silica (eluted by 0-5% MTBE/petroleum ether) to give 2,2,2-trichloroethyl 2-bromoacetate (9.5 g, 71% yield) as light yellow oil. ¹H NMR (400 MHz, CDC₃) δ: 4.82 (s, 2H), 3.98 (s, 2H).

### Step 2

A mixture of 3-((1-(4-(trifluoromethyl)phenyl)cyclobutoxy)carbonyl)but-3-enoic acid (300 mg, 0.9 mmol), 2,2,2-trichloroethyl 2-bromoacetate (245 mg, 0.915 mmol) and K₂CO₃ (152 mg, 1.1 mmol) in acetone (3 mL) was stirred at room temperature overnight. The reaction mixture was filtered, the filtrate was concentrated under reduced pressure, and the residue was purified by flash column chromatography on silica (0-10% MTBE/petroleum ether) to give 4-(2-oxo-2-(2,2,2-trichloroethoxy)ethyl) 1-(1-(4-(trifluoromethyl)phenyl)cyclobutyl) 2-methylenesuccinate (200 mg, 42% yield) as a yellow oil. LCMS m/z 539.7 (M+Na)⁺ (ES⁺).

### Step 3

A mixture of 4-(2-oxo-2-(2,2,2-trichloroethoxy)ethyl) 1-(1-(4-(trifluoromethyl)phenyl)cyclobutyl) 2-methylenesuccinate (200 mg, 0.4 mmol), zinc powder (127 mg, 2.0 mmol) and NH₄OAc (300 mg, 3.9 mmol) in THF (1.5 mL) and H₂O (0.5 mL) was stirred at room temperature for 2 hrs. The reaction mixture was filtered and the filtrate was acidified with aq HCl (0.5 N). until pH 5, and extracted with MTBE (2×5 mL). The combined organic phases were washed with brine, dried (magnesium sulfate) and concentrated under reduced pressure. The residue was purified by prep-HPLC (Column: Waters SUNFIRE Prep C18 OBD 10 μm 19×250 mm; Flow Rate: 20 mL/min; solvent system: MeCN/(0.2% formic acid/water) MeCN gradient: 50-95%; collection wavelength: 214 nm). The fractions were concentrated under reduced pressure to remove MeCN, and the residue was lyophilized to give 2-(3-((1-(4-(trifluoromethyl)phenyl)cyclobutoxy)carbonyl)but-3-enoyloxy)acetic acid (55.43 mg, 36% yield) as light yellow oil. LCMS m/z 408.9 (M+Na)⁺ (ES⁺). ¹H NMR (400 MHz, DMSO-d6) δ: 13.099 (br, 1H), 7.73 (d, J=8.4 Hz, 2H), 7.65 (d, J=8.4 Hz, 2H), 6.24 (d, J=1.2 Hz, 1H), 5.89 (d, J=0.8 Hz, 1H), 4.58 (s, 2H), 3.48 (s, 2H), 2.58 (t, J=8.8 Hz, 4H), 1.98-1.95 (m, 1H), 1.80-1.75 (m, 1H).

### Example 23—3-methyl-4-oxo-4-(1-(4-(trifluoromethyl)phenyl)cyclobutoxy)but-2-enoic acid

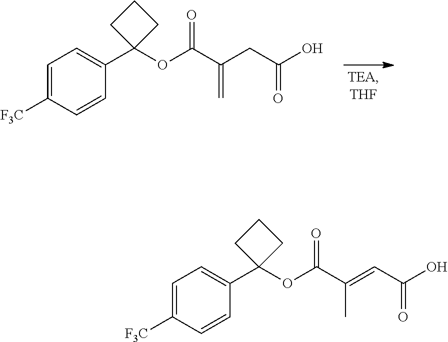 [SMILES: C=C(CC(=O)O)C(=O)OC1(c2ccc(C(F)(F)F)cc2)CCC1 ; C/C(=C\C(=O)O)C(=O)OC1(c2ccc(C(F)(F)F)cc2)CCC1]

A mixture of 3-((1-(4-(trifluoromethyl)phenyl)cyclobutoxy)carbonyl)but-3-enoic acid (Example 2, 150 mg, 0.46 mmol) in THF (2 mL) and triethylamine (1 mL) was stirred at 65° C. for 2 days. The solvent was removed under reduced pressure; the residue was diluted with MTBE (3 mL) and acidified with aq HCl (0.5 N) until pH 4-5. separated and extracted with tert-butyl methyl ether (2 20×3 mL). The combined organic phases were washed with brine, dried (magnesium sulfate) and concentrated under reduced pressure. The residue was purified by prep-HPLC (Column: Waters X-Bridge C18 OBD 10 μm 19×250 mm; Flow Rate: 20 mL/min; solvent system: MeCN/(0.2% formic acid/water) MeCN gradient:: 50-95%; collection wavelength: 214 nm). The fractions were concentrated under reduced pressure to remove MeCN, and the residue was lyophilized to give 3-methyl-4-oxo-4-(1-(4-(trifluoromethyl)phenyl)cyclobutoxy)but-2-enoic acid (45.2 mg, 31% yield) as white solid. ¹H NMR (400 MHz, DMSO-d6) δ: 13.02 (br, 1H), 7.75 (d, J=8.4 Hz, 2H), 7.69 (d, J=8.4 Hz, 2H), 6.70 (m, 1H), 2.65-2.62 (m, 4H), 2.12 (d, J=1.2 Hz, 3H), 2.02-1.91 (m, 1H), 1.82-1.70 (m, 1H).

### Biological Example 1—THP-1 AlphaLISA IL-1β and IL-6 Cytokine Assay

Measuring Inhibitory Effects on IL-1β, and IL-6 Cytokine Output from THP-1s

The cytokine inhibition profiles of compounds of formula (I) were determined in a differentiated THP-1 cell assay. All assays were performed in RPMI-1640 growth medium (Gibco), supplemented with 10% fetal bovine serum (FBS; Gibco), 1% penicillin-streptomycin and 1% sodium pyruvate unless specified otherwise. The IL-1β and IL-6 cytokine inhibition assays were each run in a background of differentiated THP-1 cells as described below. All reagents described were from Sigma-Aldrich unless specified otherwise. Compounds were prepared as 10 mM DMSO stocks.

### Assay Procedure

THP-1 cells were expanded as a suspension up to 80% confluence in appropriate growth medium. Cells were harvested, suspended, and treated with an appropriate concentration of phorbol 12-myristate 13-acetate (PMA) over a 72 hr period (37° C./5% CO₂).

Following 72 hrs of THP-1 cell incubation, cellular medium was removed and replaced with fresh growth media containing 1% of FBS. Working concentrations of compounds were prepared separately in 10% FBS treated growth medium and pre-incubated with the cells for 30 minutes (37° C./5% CO₂). Following the 30 minute compound pre-incubation, THP-1s were treated with an appropriate concentration of LPS and the THP-1s were subsequently incubated for a 24 hr period (37° C./5% CO₂). An appropriate final concentration of Nigericin was then dispensed into the THP-1 plates and incubated for 1 hour (37° C./5% CO₂) before THP-1 supernatants were harvested and collected in separate polypropylene 96-well holding plates.

Reagents from each of the IL-1β and IL-6 commercial kits (Perkin Elmer) were prepared and run according to the manufacturer's instructions. Subsequently, fluorescence signal detection in a microplate reader was measured (EnVision® Multilabel Reader, Perkin Elmer).

Percentage inhibition was calculated per cytokine by normalizing the sample data to the high and low controls used within each plate (+/−LPS respectively). Percentage inhibition was then plotted against compound concentration and the 50% inhibitory concentration (IC₅₀) was determined from the resultant concentration-response curve.

The data for all compounds of formula (I) tested in this assay are presented in Table 1 below. Dimethyl itaconate and 1-monomethyl itaconate were included as comparator compounds.

TABLE 1
THP-1 cell IL-1β and IL-6 IC₅₀ values (μM)

| Compound | IL-1β (IC₅₀) | IL-6 (IC₅₀) |
|---|---|---|
| dimethyl itaconate | >100 | >100 |
| 1-monomethyl itaconate | >100 | >100 |
| Example 1 | 5.4 | 54.8 |
| Example 2 | 4.4 | >100 |
| Example 3 | 4.8 | 53.7 |
| Example 4 | 9.2 | >100 |
| Example 5 | 9.0 | >31.6 |
| Example 6 | 9.5 | >100 |
| Example 7 | 7.9 | 65.6 |
| Example 8 | 7.6 | 10.8 |
| Example 9 | 12.6 | 11.1 |
| Example 10 | 60.0 | 13.8 |
| Example 11 | 9.9 | 13.2 |
| Example 12 | 3.1 | 4.0 |
| Example 13 | 6.5 | 8.1 |
| | 8.5* | |
| Example 14 | 5.2 | 3.9 |
| Example 15 | 5.3 | 7.3 |
| Example 16 | 5.9 | 5.3 |
| Example 17 | 15.3 | 12.4 |
| Example 18 | 5.2 | 3.8 |
| Example 19 | 2.5 | 3.8 |
| Example 20 | 2.9 | 2.6 |
| Example 21 | 7.2 | 3.9 |
| Example 22 | 15.2 | >31.6 |
| Example 23 | 4.0 | >100 |

*repeated value

These results reveal that compounds of formula (I) are expected to have anti-inflammatory activity as shown by their IC₅₀ values for inhibition of IL-1β and IL-6 release in this assay. All compounds of the invention tested exhibited improved IL-1β lowering properties (IC₅₀ values) compared with dimethyl itaconate and 1-monomethyl itaconate. Preferred compounds of the invention tested exhibited improved IL-1β and IL-6 lowering properties (IC₅₀ values) compared with dimethyl itaconate and 1-monomethyl itaconate

### Biological Example 2—NRF2 Activation Assay

### Measuring Compound Activation Effects on the Anti-Inflammatory Transcription Factor NRF2 in DiscoverX PathHunter NRF2 Translocation Kit

Potency and efficacy of compounds of formula (I) against the target of interest to activate NRF2 (nuclear factor erythroid 2-related factor 2) were determined using the PathHunter NRF2 translocation kit (DiscoverX). The NRF2 translocation assay was run using an engineered recombinant cell line, utilising enzyme fragment complementation to determine activation of the Keap1-NRF2 protein complex and subsequent translocation of NRF2 into the nucleus. Enzyme activity was quantified using a chemiluminescent substrate consumed following the formation of a functional enzyme upon PK-tagged NRF2 translocation into the nucleus.

Additionally, a defined concentration of dimethyl fumarate was used as the ‘High’ control to normalise test compound activation responses.

### Assay Procedure

U2OS PathHunter eXpress cells were thawed from frozen prior to plating. Following plating, U2OS cells were incubated for 24 hrs (37° C./5% CO₂) in commercial kit provided cell medium.

Following 24 hrs of U2OS incubation, cells were directly treated with an appropriate final concentration of compound.

Following compound treatment, the U2OS plates were incubated for a further 6 hours (37° C./5% CO₂) before detection reagent from the PathHunter NRF2 commercial kit was prepared and added to test plates according to the manufacturer's instructions. Subsequently, the luminescence signal detection in a microplate reader was measured (PHERAstar®, BMG Labtech).

Percentage activation was calculated by normalising the sample data to the high and low controls used within each plate (+/−DMF). Percentage activation/response was then plotted against compound concentration and the 50% activation concentration (EC₅₀) was determined from the plotted concentration-response curve.

The data for all compounds of formula (I) tested in this assay are presented in Table 2 below.

Dimethyl itaconate and 1-monomethyl itaconate were included as comparator compounds.

TABLE 2
NRF2 activation

| | −GSH | | +GSH | |
|---|---|---|---|---|
| Compound | EC₅₀ (μM) | Eₘₐₓ (%) | EC₅₀ (μM) | Eₘₐₓ (%) |
| dimethyl itaconate | 21.7 | 126 | 56.4 | 77 |
| 1-monomethyl | 30.1 | 44 | NT* | NT |
| itaconate | | | | |
| Example 2 | >100 | 26 | >100 | 14 |
| Example 8 | 51 | 51 | >100 | 38 |
| Example 11 | 33.1 | 117 | 45.7 | 100 |
| Example 13 | 38.8 | 174 | 48.0 | 84 |
| Example 14 | 14.6 | 80 | 49.4 | 95 |
| Example 15 | 26.3 | 131 | 51.3 | 86 |
| Example 18 | 14.7 | 90 | 39.4 | 70 |
| Example 19 | 40.2 | 210 | 50.7 | 102 |
| Example 20 | 14.4 | 101 | 31.4 | 63 |
| Example 21 | 38.0 | 184 | 55.0 | 86 |
| Example 22 | 40.0 | 100 | 41.1 | 45 |
| Example 23 | 2.8 | 39 | >100 | 4 |

NT = not tested in this assay

For the most part, the compounds in Table 2 display relatively low NRF2 activating effects compared with the controls, as demonstrated by their EC₅₀ and/or Eₘₐₓ values for NRF2 activation, indicating that the ULP˜-lowering effect may not be solely a consequence of NRF2 activation. Thus, these compounds are expected to display reduced effects resulting from NRF2 activation.

### Biological Example 3—Hepatocyte Stability Assay

Defrosted cryo-preserved hepatocytes (viability >70%) were used to determine the metabolic stability of a compound via calculation of intrinsic clearance (CIᵢₙₜ; a measure of the removal of a compound from the liver in the absence of blood flow and cell binding). Clearance data are particularly important for in vitro work as they can be used in combination with in vivo data to predict the half-life and oral bioavailability of a drug.

The metabolic stability in hepatocytes assay involved a time-dependent reaction using both positive and negative controls. The cells must be pre-incubated at 37° C. then spiked with test compound (and positive control); samples taken at pre-determined time intervals were analysed to monitor the change in concentration of the initial drug compound over 60 minutes. A buffer incubation reaction (with no hepatocytes present) acted as a negative control and two cocktail solutions, containing compounds with known high and low clearance values (verapamil/7-hydroxycoumarin and propranolol/diltiazem), acted as positive controls.
1. The assay was run with a cell concentration of 0.5×10⁶ cells/mL in Leibovitz buffer.
2. All compounds and controls were run in duplicate.
3. Compound concentration was 10 μM.
4. All compounds and controls were incubated with both cells and buffer to show turnover is due to hepatic metabolism.
5. All wells on the incubation plate had 326.7 μL of either cells or buffer added.
6. Prior to assay, cell and buffer-only incubation plates were preincubated for 10 mins at 37° C.
7. The assay was initiated by adding compounds, 3.3 μL of 1 mM in 10% DMSO-90% Buffer; final DMSO concentration is 0.1%.
8. Samples were taken at regular timepoints (0, 5, 10, 20, 40, 60 min) until 60 mins.
9. Sample volume was 40 μL and added to 160 μL of crash solvent (acetonitrile with internal standard) and stored on ice.
10. At the end of the assay, the crash plates were centrifuged at 3500 rpm for 20 mins at 4° C.
11. 80 μL of clear supernatant was removed and mixed with 80 μL of deionised water before being analysed by LC-MS/MS.

Raw LC-MS/MS data were exported to, and analysed in, Microsoft Excel for determination of intrinsic clearance. The percentage remaining of a compound was monitored using the peak area of the initial concentration as 100%. Intrinsic clearance and half-life values were calculated using a graph of the natural log of percentage remaining versus the time of reaction in minutes. Half-life (min) and intrinsic clearance (CIᵢₙₜ in μL min⁻¹ 10⁻⁶ cells) values were calculated using the gradient of the graph (the elimination rate constant, k) and Equations 1 and 2.

$${t}_{\frac{1}{2}} = \frac{ln 2}{k} \qquad \{ Equation 1 \}$$

$${CI}_{i n t} = ( \frac{ln 2}{{t}_{\frac{1}{2}}} ) \times ( \frac{350}{0.175} ) \qquad \{ Equation 2 \}$$

A number of compounds of formula (I) were tested in this assay, and the results are shown in Table 3 below. 4-Octyl itaconate was included as a comparator compound.

TABLE 3
Hepatocyte stability

| Compound | Species | CIᵢₙₜ (μL min⁻¹ 10⁻⁶ cells) | T½ (min) |
|---|---|---|---|
| 4-octyl itaconate | Human | 401 | 4 |
| | Mouse | 351 | 4 |
| Example 1 | Human | <3 | >460 |
| | Mouse | <3 | >460 |
| Example 2 | Human | 9 | 126 |
| | Mouse | <3 | >460 |
| Example 3 | Human | <3 | >460 |
| | Mouse | <3 | >460 |
| Example 4 | Human | 6 | 314 |
| | Mouse | 8 | 219 |
| Example 5 | Human | <3 | >460 |
| | Mouse | <3 | >460 |
| Example 6 | Human | 13 | 143 |
| | Mouse | <3 | >460 |
| Example 7 | Human | 8 | 219 |
| | Mouse | 11 | 118 |
| Example 8 | Human | >460 | <3 |
| | Mouse | >460 | <3 |
| Example 9 | Human | 27 | 44 |
| | Mouse | 449 | 3 |
| Example 10 | Human | 98 | 12 |
| | Mouse | >460 | <3 |
| Example 11 | Human | 9 | 139 |
| | Mouse | 162 | 9 |
| Example 12 | Human | 59 | 25 |
| | Mouse | 138 | 9 |
| Example 13 | Human | 126 | 12 |
| | Mouse | 516 | 4 |
| Example 14 | Human | 40 | 38 |
| | Mouse | 108 | 12 |
| Example 15 | Human | 92 | 14 |
| | Mouse | 508 | 3 |
| Example 16 | Human | 114 | 11 |
| | Mouse | >460 | <3 |
| 4-octyl itaconate | Human | 401 | 4 |
| | Mouse | 351 | 4 |
| Example 18 | Human | 31 | 52 |
| | Mouse | 92 | 14 |
| Example 19 | Human | 32 | 48 |
| | Mouse | 161 | 13 |
| Example 20 | Human | 85 | 18 |
| | Mouse | 668 | 3 |
| Example 21 | Human | 37 | 42 |
| | Mouse | 100 | 21 |
| Example 22 | Human | 7 | 204 |
| | Mouse | 392 | 4 |
| Example 23 | Human | <3 | >460 |
| | Mouse | <3 | >460 |

The results indicate that the compounds of the invention, at least those of Table 3, are expected to have acceptable or improved metabolic stabilities, as shown by their intrinsic clearance (CIᵢₙₜ) and half-life (T_{1/2}) values, in this assay. All compounds in Table 3, except Example 8, were more stable, i.e., they exhibited lower intrinsic clearance (CIᵢₙₜ) and longer half-life (T_{1/2}) values compared with 4-octyl itaconate in at least human or mouse species. Preferred compounds exhibited lower intrinsic clearance (CIᵢₙₜ) and longer half-life (T_{1/2}) values compared with 4-octyl itaconate in both human and mouse species.

### REFERENCES

The following publications cited in this specification are herein incorporated by reference in their entirety.
Ackermann et al. Proc. Soc. Exp. Bio. Med. 1949, 72(1), 1-9.
Andersen J. L. et al. Nat. Commun. 2018, 9, 4344.
Angiari S. and O'Neill L. A. Cell Res. 2018, 28, 613-615.
Bagavant G. et al. Indian J. Pharm. Sci. 1994, 56, 80-85.
Bambouskova M. et al. Nature 2018, 556, 501-504.
Blewett M. M. et al. Sci. Sign. 2016, 9 (445), rs10; 6.
Brennan M. S. et al. PLoS One 2015, 10, e0120254.
BrCick J. et al. Exp. Dermatol. 2018, 27, 611-624.
Cocco M. et al. J. Med. Chem. 2014, 57, 10366-10382.
Cocco M. et al. J. Med. Chem. 2017, 60, 3656-3671.
Cordes T. et al. J. Biol. Chem. 2016, 291, 14274-14284.
Cordes T. et al. Mol. Metab. 2020, 32, 122-135.
Daly R. et al. Metabolites, 2020 Jun. 10; 10(6), 241.
Daniels B. P. et al. Immunity 2019, 50(1), 64-76.e4.
Dibbert S. et al. Arch. Dermatol. Res. 2013, 305, 447-451.
ElAzzouny M. et al. J. Biol. Chem. 2017, 292, 4766-4769.
Gillard G. O. et al. J. Neuroimmunol. 2015, 283, 74-85.
Gu L. et al. Immunol. Cell Biol. 2020; 98, 229-241.
Hanke T. et al. Pharmacol. Therapeut. 2016, 157, 163-187.
Hunt T. et al. Consortium of Multiple Sclerosis Centers 2015 Annual Meeting, 27-30 May 2015, Indianapolis, IN, USA: Poster DX37.
Kornberg M. D. et al. Science 2018, 360, 449-453.
Lampropoulou V. et al. Cell Metab. 2016, 24, 158-166.
Lehmann J. C. U. et al. J. Invest. Dermatol. 2007, 127, 835-845.
Liao S.-T. et al. Nat. Commun. 2019, 10(1), 5091.
Liu H. et al. Cell Commun. Signal. 2018, 16, 81.
McGuire V. A. et al. Sci. Rep. 2016, 6, 31159.
Michelucci A. et al. Proc. Natl. Acad. Sci. USA 2013, 110, 7820-7825.
Mills E. A. et al. Front. Neurol. 2018, 9, 5.
Mills E. L. et al. Cell 2016, 167, 457-470.
Mills E. L. et al. Nature 2018, 556, 113-117.
Mrowietz U. et al. Trends Pharmacol. Sci. 2018, 39, 1-12.
Müller S. et al. J. Dermatol. Sci. 2017, 87, 246-251.
Murphy M. P. and O'Neill L. A. J. Cell 2018, 174, 780-784.
O'Neill L. A. J. and Artyomov M. N. Nat. Rev. Immunol. 2019 273-281.
Olagnier D. et al. Nat. Commun. 2018, 9, 3506.
Schmidt T. J. et al. Bioorg. Med. Chem. 2007, 15, 333-342.
Shan Q. et al. Biochem. Biophys. Res. Commun. 2019, 517, 538-544.
Straub R. H. and Schradin C. Evol. Med. Public Health 2016, 1, 37-51S.
Straub R. H. and Cutolo M. Rheumatology 2016, 55 (Suppl. 2), ii6-ii14.
Sun X. et al., FASEB J. 2019, 33, 12929-12940.
Tang C. et al. Cell Physiol. Biochem. 2018, 51, 979-990.
Tang C. et al. Biochem. Biophys. Res. Commun. 2019, 508, 921-927.
Tang H. et al. Biochem. Biophys. Res. Commun. 2008, 375, 562-565.
Tian et al. Eur. J. Pharmacol. 2020, 873, 172989.
van der Reest J. et al. Nat. Commun. 2018, 9, 1581.
von Glehn F. et al. Mult. Scler. Relat. Disord. 2018, 23, 46-50.
Yi F. et al. Hepatology 2020, 873, 172989.
Yu X.-H. et al. Immunol. Cell Biol. 2019, 97, 134-141.
Zhang S. et al. Bioorg. Med. Chem. 2012, 20, 6073-6079.
Zhang D. et al. Int. Immunopharmacol. 2019, 77, 105924.
Zhao C. et al. Microb. Pathogen. 2019, 133, 103541.
Zhao G. et al. Biochem. Biophys. Res. Commun. 2014, 448, 303-307.

### MISCELLANEOUS

All references referred to in this application, including patent and patent applications, are incorporated herein by reference to the fullest extent possible.

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

1: A compound of formula (I): 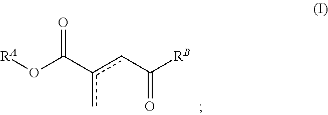
wherein the moiety: 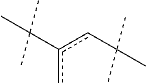
represents 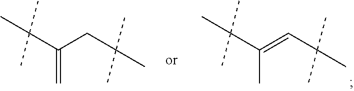
R^{A} is: 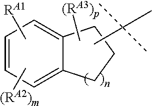
wherein:
R^{A1} is selected from the group consisting of H, halo, C₁₋₄ alkyl, C₁₋₄ haloalkyl, C₁₋₄ alkoxy, C₁₋₄ haloalkoxy, —SC₁₋₄ alkyl, —SC₁₋₄ haloalkyl and SF₅;
R^{A2} is selected from the group consisting of halo, C₁₋₄ alkyl, C₁₋₄ haloalkyl, C₁₋₄ alkoxy and C₁₋₄ haloalkoxy;
R^{A3} is C₁₋₂ alkyl;
m is 0, 1 or 2;
n is 1 or 2; and
p is 0, 1, 2, 3 or 4; or R^{A} is: 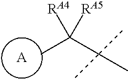
wherein:
R^{A4} is H, C₁₋₄ alkyl, C₁₋₄haloalkyl or C≡CH; and
R^{A5} is H, C₁₋₄ alkyl or C₁₋₄ haloalkyl;
or R^{A4} and R^{A5} join to form a C₃₋₆ cycloalkyl ring or a 4-6 membered heterocyclic ring wherein the C₃₋₆ cycloalkyl ring is optionally substituted by one or more R^{A6}, wherein R^{A6} is independently selected from the group consisting of fluoro, methyl and cyano, or two R^{A6} groups which are attached to the same carbon atom join to form a C₃₋₄ cycloalkyl ring;
A is phenyl or 5-6-membered heteroaryl wherein when A is phenyl or 6-membered heteroaryl A is optionally substituted by one or more R^{A7}, and when A is 5-membered heteroaryl A is substituted by one or more R^{A7}, wherein when A is phenyl substituted by one or more R^{A7}, one R^{A7} is in the 4-position with respect to C(R^{A4})(R^{A5});
R^{A7} is independently selected from the group consisting of halo, C₁₋₄ alkyl, C₁₋₄ haloalkyl, C₁₋₄ haloalkoxy, SF₅, SC₁₋₄ alkyl and SC₁₋₄ haloalkyl; and
R^{B} is NR^{B1}R^{B2} or OR^{B3};
wherein:
R^{B1} and R^{B2} are independently selected from the group consisting of H, C₁₋₄ alkyl and C₁₋₄ haloalkyl and 4-7-membered heterocyclyl wherein R^{B1} and/or R^{B2} are optionally substituted by one or more R^{B4} wherein R^{B4} is selected from the group consisting of C₁₋₂ alkyl, NH₂, N(C₁₋₂ alkyl)₂, hydroxy, oxo, 5-7-membered heterocyclyl and 5-6-membered heteroaryl optionally substituted by C₁₋₂ alkyl; and
R^{B3} is selected from the group consisting of H, C₁₋₄ alkyl, C₁₋₄ haloalkyl, CH₂COOH, CH₂CH₂COOH, CH₂tetrazolyl and CH₂CH₂tetrazolyl, wherein R^{B3} is optionally substituted on an available carbon atom by one or more R^{B3′} wherein R^{B3′} is selected from the group consisting of difluoromethyl, trifluoromethyl and methyl; and/or wherein R^{B3} is optionally substituted by two R^{B3′} groups which are attached to the same carbon atom and join to form a C₃₋₆ cycloalkyl or a 4-6-membered heterocyclyl ring;
or a pharmaceutically acceptable salt and/or solvate thereof.

2: The compound, pharmaceutically acceptable salt and/or solvate thereof according to claim 1 wherein the moiety: 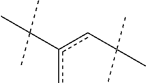
represents

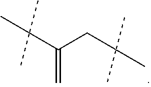

3. (canceled)

4. The compound, pharmaceutically acceptable salt and/or solvate thereof according to claim 1 wherein R^{A} is: 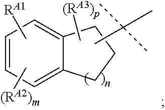
wherein R^{A1}, R^{A2}, R^{A3}, m, n and p are defined in claim 1.

5: The compound, pharmaceutically acceptable salt and/or solvate thereof according to claim 4 wherein R^{A1} is C₁₋₄ haloalkyl.

6: The compound, pharmaceutically acceptable salt and/or solvate thereof according to claim 4 wherein m is 0.

7: The compound, pharmaceutically acceptable salt and/or solvate thereof according claim 4 wherein n is 1.

8: The compound, pharmaceutically acceptable salt and/or solvate thereof according to claim 4 wherein p is 0.

9: The compound, pharmaceutically acceptable salt and/or solvate thereof according to claim 1 wherein R^{A} is: 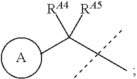
wherein A, R^{A4} and R^{A5} are defined in claim 1.

10: The compound, pharmaceutically acceptable salt and/or solvate thereof according to claim 9 wherein R^{A4} is C₁₋₄ alkyl such as methyl.

11: The compound, pharmaceutically acceptable salt and/or solvate thereof according to claim 9 wherein R^{A5} is H.

12: The compound, pharmaceutically acceptable salt and/or solvate thereof according to claim 9 wherein when R^{A4} and R^{A5} are different, the carbon atom to which R^{A4} and R^{A5} are attached has the following stereochemistry:

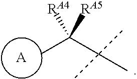

13: The compound, pharmaceutically acceptable salt and/or solvate thereof according to claim 9 wherein R^{A4} and R^{A5} join to form a C₃₋₆ cycloalkyl ring or a 4-6 membered heterocyclic.

14: The compound, pharmaceutically acceptable salt and/or solvate thereof according to claim 13 wherein the C₃₋₆ cycloalkyl ring is not substituted; or
wherein the C₃₋₆ cycloalkyl ring is substituted by one or more R^{A6}, wherein R^{A6} is independently selected from the group consisting of fluoro, methyl and cyano, or two R^{A6} groups which are attached to the same carbon atom join to form a C₃₋₄ cycloalkyl ring.

15-16. (canceled)

17: The compound, pharmaceutically acceptable salt and/or solvate thereof according to claim 9 wherein A is phenyl, thienyl or pyridinyl.

18. (canceled)

19: The compound, pharmaceutically acceptable salt and/or solvate thereof according to claim 9 wherein A is unsubstituted phenyl or unsubstituted 6-membered heteroaryl.

20: The compound, pharmaceutically acceptable salt and/or solvate thereof according to claim 9 wherein A is substituted by one or more, wherein R^{A7} is defined in claim 1, and wherein when A is phenyl substituted by one or more R^{A7}, one R^{A7} is in the 4-position with respect to C(R^{A4})(R^{A5}).

21: The compound, pharmaceutically acceptable salt and/or solvate thereof according to claim 20 wherein R^{A7} is halo or C₁₋₄ haloalkyl.

22. (canceled)

23: The compound, pharmaceutically acceptable salt and/or solvate thereof according to claim 1 wherein R^{B} is NR^{B1}R^{B2}.

24-36. (canceled)

37: The compound, pharmaceutically acceptable salt and/or solvate thereof according to claim 1 wherein R^{B} is OR^{B3}.

38-45. (canceled)

46: The compound according to claim 1, which is selected from the list consisting of:
(S)-3-((1-(4-(trifluoromethyl)phenyl)ethoxy)carbonyl)but-3-enoic acid;
3-((1-(4-(trifluoromethyl)phenyl)cyclobutoxy)carbonyl)but-3-enoic acid;
3-((1-(3-fluoro-4-(trifluoromethyl)phenyl)cyclobutoxy)carbonyl)but-3-enoic acid;
3-(((3-(4-(trifluoromethyl)phenyl)oxetane-3-yl)oxy)carbonyl)but-3-enoic acid;
3-((1-(5-(trifluoromethyl)pyridin-2-yl)cyclobutoxy)carbonyl)but-3-enoic acid;
3-((1-(5-(trifluoromethyl)thiophen-2-yl)cyclobutoxy)carbonyl)but-3-enoic acid;
3-(((6-(trifluoromethyl)-1,2,3,4-tetrahydronaphthalen-1-yl)oxy)carbonyl)but-3-enoic acid;
4-methyl 1-(1-(4-(trifluoromethyl)phenyl)cyclobutyl) 2-methylenesuccinate;
1-(4-(trifluoromethyl)phenyl)cyclobutyl 4-(methylamino)-2-methylene-4-oxobutanoate;
1-(4-(trifluoromethyl)phenyl)cyclobutyl 4-(dimethylamino)-2-methylene-4-oxobutanoate;
1-(4-(trifluoromethyl)phenyl)cyclobutyl 4-amino-2-methylene-4-oxobutanoate;
1-(4-(trifluoromethyl)phenyl)cyclobutyl 4-((2-(dimethylamino)ethyl)amino)-2-methylene-4-oxobutanoate;
1-(4-(trifluoromethyl)phenyl)cyclobutyl 2-methylene-4-((2-morpholinoethyl)amino)-4-oxobutanoate;
1-(4-(trifluoromethyl)phenyl)cyclobutyl 2-methylene-4-((1-methylpiperidin-4-yl)amino)-4-oxobutanoate;
1-(4-(trifluoromethyl)phenyl)cyclobutyl 4-(((1H-imidazol-2-yl)methyl)amino)-2-methylene-4-oxobutanoate;
1-(4-(trifluoromethyl)phenyl)cyclobutyl 4-(((1-methyl-1H-imidazol-2-yl)methyl)amino)-2-methylene-4-oxobutanoate;
1-(4-(trifluoromethyl)phenyl)cyclobutyl 4-((1-amino-3-hydroxy-1-oxopropan-2-yl)amino)-2-methylene-4-oxobutanoate;
1-(4-(trifluoromethyl)phenyl)cyclobutyl 4-((1,1-dioxidothietan-3-yl)amino)-2-methylene-4-oxobutanoate;
1-(4-(trifluoromethyl)phenyl)cyclobutyl 4-((2-hydroxyethyl)amino)-2-methylene-4-oxobutanoate;
1-(4-(trifluoromethyl)phenyl)cyclobutyl 2-methylene-4-oxo-4-((tetrahydro-2H-pyran-4-yl)amino)butanoate;
1-(4-(trifluoromethyl)phenyl)cyclobutyl 4-((2-amino-2-oxoethyl)amino)-2-methylene-4-oxobutanoate;
2-((3-((1-(4-(trifluoromethyl)phenyl)cyclobutoxy)carbonyl)but-3-enoyl)oxy)acetic acid; and
3-methyl-4-oxo-4-(1-(4-(trifluoromethyl)phenyl)cyclobutoxy)but-2-enoic acid;
or a pharmaceutically acceptable salt and/or solvate of any one thereof.

47: A pharmaceutical composition comprising a compound, pharmaceutically acceptable salt and/or solvate thereof according to claim 1.

48-50. (canceled)

51: A method of treating or preventing an inflammatory disease or a disease associated with an undesirable immune response, which comprises administering a compound, pharmaceutically acceptable salt and/or solvate thereof according to claim 1.

52-56. (canceled)

57: The method according to claim 51, wherein the inflammatory disease or disease associated with an undesirable immune response is selected from the group consisting of rheumatoid arthritis, psoriatic arthritis, ankylosing spondylitis, systemic lupus erythematosus, multiple sclerosis, psoriasis, Crohn's disease, ulcerative colitis, uveitis, cryopyrin-associated periodic syndromes, Muckle-Wells syndrome, juvenile idiopathic arthritis, chronic obstructive pulmonary disease and asthma.

58-68. (canceled)
